(19) United States
(12) Patent Application Publication
(10) Pub. No.: US 2025/0005677 A1
(43) Pub. Date: Jan. 2, 2025

(54) A SYSTEM AND METHOD FOR DETERMINING ANIMAL INSURANCE PARAMETERS

(71) Applicant: IDENTIGEN LIMITED, Dublin (IE)

(72) Inventors: Yuval RAPAPORT-ROM, Haifa (IL); Robert William MITCHELL, Guildford (GB); Hubert BOURKE-BORROWES, London (GB); Matteo RATTI, Piacenza (IT)

(73) Assignee: IDENTIGEN LIMITED, Dublin (IE)

(21) Appl. No.: 18/701,373

(22) PCT Filed: Oct. 14, 2022
(86) PCT No.: PCT/EP2022/078713

§ 371 Date: Apr. 15, 2024

(30) Foreign Application Priority Data
Oct. 15, 2021 (IL) 287303

(51) Int. Cl. G06Q 40/08; G06Q 10/0637; G16H 50/30

(52) U.S. Cl. CPC G06Q 40/08; G06Q 10/06375; G16H 50/30

(57) ABSTRACT

A system for determining insurance parameters, the system comprising: one or more monitoring devices configured to monitor parameters of members of an animal population; a data repository comprising two or more records, each (i) being associated with a respective member of the members, and (ii) including one or more monitored parameters of the respective member as monitored by at least one of the monitoring devices over time; and a processing circuitry configured to: obtain at least a subset of the records, the subset being associated with a group of given members of the members; determine one or more GHGE affecting parameters based on the subset; estimate an amount of GHGE in an environment housing the animal population utilizing the GHGE affecting parameters; and determine one or more insurance parameters, for insuring at least one animal of the animal population, based on the estimated amount.

### TECHNICAL FIELD

The presently disclosed subject matter relates to animal insurance parameters determination and/or verification of an identity of an animal.

### BACKGROUND

Many transactions require animals to be identified, and that the animal identity can be verified when needed. It is a well-known convention that uncertainty has a negative effect on transaction costs. One type of uncertainty in transactions that involve specific animals is the uncertainty of the animal's identity. The risk of fraudulent activities associated with falsely identifying an animal in transactions involving animals (such as animal insurance, loans that involve animal as collateral, sale of an animal, etc.) results in high transaction costs.

An animal can be identified in many ways, some of which are more accurate and/or secure than others. For example, an animal can be visually identified by a human or by a computerized system (e.g., using image analysis), the visual identification can optionally be made using a marking painted on the animal. The animal can be also identified using an electronic identification tag attached to the animal or using Deoxyribonucleic acid (DNA) analysis, and more. Clearly, visually identifying an animal, even using a marker painted on its body, is not as accurate and secure as identifying the animal using DNA analysis (e.g. DNA sequencing) which uniquely identifies the animal (disregarding cloned animals which share the same DNA), and clearly it is more prawn to errors or fraud attempts.

In order to deal with fraud and reduce the uncertainty associated with an animal's identity in transactions that involve an animal, it is desirable to verify the identity of the animal. Looking at insurance as an example, insurance fraud includes acts made by claimants that are aimed at gaining benefits that the claimants are not entitled to. In such fraud, false insurance claims are filed with the fraudulent intention towards an insurance provider, in order to get undue compensation.

A specific type of insurance is animal insurance in which individual animals are insured against death, disease or other loss. In such types of insurance, which in some cases can include a considerable compensation, insurers need to verify the identity of an insured animal when a compensation triggering event occurs (e.g., the animal dies, becomes ill, or any other reason triggered the insurance policy), in order to at least reduce the risk for insurance fraud.

Currently, and especially when the insurance includes a considerable compensation, when an insurance triggering event occurs—a human evaluator is sent to validate the identity of an animal in order to prevent fraud. The identity of the animal is validated based on the skills of the human evaluator, and optionally based on images of the animal and additional non-biometric data thereof. Such human identity validation is problematic as it is prone to errors, expensive, cumbersome, and requires fast response times from all parties involved. When an insurance event occurs, the insurance owner has to report the insurance event to the insurer, and in case of a high value insurance claim—the insurer is required to send the human evaluator to validate the identity of the animal (or group of animals) and the occurrence of the insurance event (noting that in some cases the insured animal/s is/are located at an isolated location remote from available human evaluators), and the human evaluator performs the evaluation in a process that itself, as indicated herein, is not error free. Therefore, existing insurance claim validation processes are lacking.

The same applies also to collateral claims, in which an animal is used as collateral, and to animal sales, or sale of a product generated by the animal or from the animal. In order to reduce the risks and/or costs associated with fraudulent activities, the identity of the animal used as collateral, or being sold, needs to be verified.

There is thus a real need in the art for a new system and method for animal identity verification, which can enable, for example, risk mitigation and act as a disincentive for fraud. Information collected using the system and method disclosed herein can also enable stakeholders to use a wider range of information when providing services.

Animal insurance pricing can be affected by various parameters, such as animal health, animal welfare, greenhouse gas emissions in an environment housing the animal, etc.

Greenhouse gas (also referred to as GHG) absorbs Infra-Red (IR) radiation emitted from earth's surface, and redirects it back to earth's surface, causing global warming. Livestock are responsible for a considerable amount of global greenhouse gases, evaluated by some at 14.5%. In view of the significance of livestock contribution to global greenhouse gas emissions, many studies have been made, showing that improving fertility, health, feed and herd management can have a substantial contribution to reducing livestock greenhouse gas emissions, and improving the ratio of GHG emission to milk production (so as to generate less GHG per milk unit generated by a given animal population).

Nowadays, greenhouse gas emission in environments housing animal populations is mainly determined by a subjective human auditor estimating the greenhouse gas emissions, or by direct measurements acquired by dedicated GHG measurement instrumentation.

Human auditors base their GHG emission estimation on data collected in a slow and manual process that is prone to errors. As part of the GHG emission estimation process, the human auditors visit and visually inspect the facilities housing the animals, and gather various types of data on the animal population itself. In some cases, the data required for the human auditor to be able to estimate GHG emissions, or parts thereof, is not available (at times due to the fact that it is not collected). Even when such data is available, it is non-homogenous as different facility owners collect different data, using different data collection methods. Based at least in part on the human auditor's subjective impression, and based on the non-homogenous data (which may also be partial), the human auditor estimates the GHG emissions in the audited environment.

One exemplary problem of the subjectivity of an auditor is its first impression of an animal facility. For a human auditor, it may be hard to shake off a first impression of a facility. Such first impression can lead to erroneous assessment of GHG emissions in such facility. For example, if the animal facility makes a good first impression, the auditor may miss out on other problems, or give them lesser weight, or vice versa.

It can be appreciated that efforts are made to train auditors to maintain standard levels of evaluation, however interobserver reliability and consistency can be low despite training of the human auditors. Even in those cases where auditors undergo identical training, that training needs to be continuous to hold high standards of consistency, and even in these cases, the subjectiveness bias is not, and cannot, be completely irradicated.

Thus, current GHG emission estimation methods are prone to the subjectivity of the auditor. On top of that, they are also sensitive to the data collection method, sensitive to the timing, circumstances, and frequency of the inspection (lighting conditions, weather conditions, etc.), and require long gathering and processing times.

In addition, GHG emission estimations may vary between auditors, and between audits, because they are not produced in real-time, nor are they based on continuous monitoring. Human auditing is not based on continuous, or near continuous, monitoring of various parameters (e.g., parameters relating to the animals' environment, the animals' health measures, animal treatments, the weather, etc.), inter alia because it is impossible for a human to track such amounts of data, let alone track such amounts of data in a manner that will enable evaluating GHG emissions in the required speed. To the contrary, having an automatic mechanism for estimating GHG emissions, optionally in real-time or near real-time, continuously or near continuously, can enable a much more complete and more accurate GHG emission estimation that is not dependent on outdated or no-longer relevant data.

As mentioned above, GHG emissions can be measured directly using dedicated instrumentation (e.g. Respiratory Chamber, Tracer technique (SF₆), Non dispersive Infrared Methane detector, Micro-Meteorological Techniques, Laser Methane Detector (LMD), etc.), however such instrumentation is usually unavailable for livestock growers to continuously and directly measure greenhouse gas emissions in the environment in which their livestock is grown. Direct measurement of GHG emissions is also an extremely difficult task to perform when the animals are not within a closed housing in which the dedicated instrumentation can yield accurate results. For example, dedicated instrumentation cannot provide an accurate measurement when the animal population is grazing in open pasture.

In spite of the critical effect of livestock on global greenhouse gas emission, existing solutions do not offer non-biased, continuous, or near continuous, visibility to the stakeholders of this industry as to GHG emission in the environment in which the livestock is grown.

In addition, no objective GHG measure is used in order to determine animal insurance parameters.

Thus, there is a real need for a new and improved system and method for animal insurance, including a system and method that use an estimation of greenhouse gas emissions in an environment housing an animal population for determining animal insurance parameters.

### GENERAL DESCRIPTION

In accordance with a first aspect of the presently disclosed subject matter, there is provided a system for estimating greenhouse gas emission in an environment housing an animal population, the system comprising: one or more monitoring devices configured to monitor parameters of members of the animal population; a data repository comprising two or more records, each of the records (i) being associated with a respective member of the members, and (ii) including one or more monitored parameters of the respective member as monitored by at least one of the monitoring devices over time; and a processing circuitry configured to: obtain at least a subset of the records, the subset being associated with a group of given members of the members; determine one or more greenhouse gas emission affecting parameters based on the subset, wherein at least one greenhouse gas emission affecting parameter of the greenhouse gas emission affecting parameters is determined based on one or more of the monitored parameters that are monitored for at least two members of the group of given members; estimate an amount of greenhouse gas emission in the environment utilizing the greenhouse gas emission affecting parameters; and determine one or more insurance parameters, for insuring at least one animal of the animal population, based on the estimated amount.

In one embodiment of the presently disclosed subject matter and/or embodiments thereof, the insurance parameters include one or more of: insurance price, insurance period, or obligations to meet in order to be eligible for insurance.

In one embodiment of the presently disclosed subject matter and/or embodiments thereof, the estimation is based on comparison of the greenhouse gas emission affecting parameters to a given baseline.

In one embodiment of the presently disclosed subject matter and/or embodiments thereof, the baseline is geographical location specific.

In one embodiment of the presently disclosed subject matter and/or embodiments thereof, the baseline is determined based on historical values of the greenhouse gas emission affecting parameters.

In one embodiment of the presently disclosed subject matter and/or embodiments thereof, the baseline is determined using greenhouse gas emission measurements acquired using greenhouse gas emission measuring equipment.

In one embodiment of the presently disclosed subject matter and/or embodiments thereof, the greenhouse gas emission measurements are acquired from the environment.

In one embodiment of the presently disclosed subject matter and/or embodiments thereof, the processing resource is further configured to perform at least one of: (a) providing a user of the system with an indication of the estimated amount of greenhouse gas emission, or (b) suggesting one or more greenhouse gas emission reduction actions to be taken on at least part of the animal population in order to reduce the greenhouse gas emission in the environment.

In one embodiment of the presently disclosed subject matter and/or embodiments thereof, the greenhouse gas emission reduction actions include one or more of: administering a treatment to the at least part of the animal population, changing a temperature of an environment of the at least part of the animal population, changing a feed of the at least part of the animal population, or changing a schedule of the at least part of the animal population.

In one embodiment of the presently disclosed subject matter and/or embodiments thereof, the greenhouse gas emission affecting parameters include one or more of the following: (a) a welfare score indicative of a health state of the group of given members, (b) Heat Detection Rate (HDR) calculated for the group of given members, (c) a conception rate calculated for the group of given members, (d) a health score calculated for the group of given members, (e) rumination consistency calculated for the group of given members, or (f) rumination time heterogeneity calculated for the group of given members.

In one embodiment of the presently disclosed subject matter and/or embodiments thereof, the processing circuitry is further configured to calculate, based on the subset of the records, for each given member of the group of given members, at least one of: (a) an animal health score indicative of a health state of the respective member, (b) an animal natural living score, indicative of compliance of a behavior pattern of the respective member with a desired natural behavior pattern, or (c) an animal affectivity/happiness score, indicative of compliance of an affectivity/happiness measure of the respective member with a desired affectivity/happiness measure, the affectivity/happiness measure being determined based on one or more of the following monitored parameters, being affectivity/happiness parameters: (1) respiration level of the given member, (2) percentage of rumination time within a fourth time period of the given member, or (3) percentage of feeding time within a fifth time period of the given member; and wherein: the health score is calculated based on the animal health scores calculated for the group of given members; and the welfare score is calculated based on the at least one of: (a) the health score, (b) the animal natural living scores calculated for the group of given members, or (c) the animal affectivity/happiness scores calculated for the group of given members.

In one embodiment of the presently disclosed subject matter and/or embodiments thereof, the monitored parameters include one or more of the following behavioral parameters: (a) percentage of movement time within a first time period of the given member, (b) percentage of feeding time within a second time period of the given member or (c) percentage of social behavior time within a third time period of the given member; and wherein the animal natural living score for the given member is determined based on the behavioral parameters.

In one embodiment of the presently disclosed subject matter and/or embodiments thereof, the natural living score for the given member is determined based on consistency of values of at least some of the behavioral parameters over time.

In one embodiment of the presently disclosed subject matter and/or embodiments thereof, the consistency of the values is measured against reference parameters.

In one embodiment of the presently disclosed subject matter and/or embodiments thereof, the reference parameters are measured from reference animals.

In one embodiment of the presently disclosed subject matter and/or embodiments thereof, the affectivity/happiness score for the given member is determined based on consistency of values of at least some of the affectivity/happiness parameters over time.

In one embodiment of the presently disclosed subject matter and/or embodiments thereof, the consistency of the values is measured against reference parameters.

In one embodiment of the presently disclosed subject matter and/or embodiments thereof, the reference parameters are measured from reference animals.

In one embodiment of the presently disclosed subject matter and/or embodiments thereof, the welfare score is calculated based on a variation between the affectivity/happiness scores of the group of given members.

In one embodiment of the presently disclosed subject matter and/or embodiments thereof, the records also include one or more environmental parameters, indicative of the state of the environment of the respective member and wherein the determination of (a) the animal natural living score of the respective member or (b) of the animal affectivity/happiness score of the respective member, is also based on the environmental parameters.

In one embodiment of the presently disclosed subject matter and/or embodiments thereof, the monitoring devices include one or more of: an accelerometer, a temperature sensor, a location sensor, a pedometer, or a heart rate sensor.

In one embodiment of the presently disclosed subject matter and/or embodiments thereof, at least some of the records further include descriptive data associated with the respective member, wherein the descriptive data is not obtained from the monitoring devices.

In one embodiment of the presently disclosed subject matter and/or embodiments thereof, the descriptive data includes one or more of: age of the respective member, sex of the respective member, treatment history of the respective member, or genetic information associated with the respective member.

In one embodiment of the presently disclosed subject matter and/or embodiments thereof, the at least a subset of the records are all the records of the animal population.

In one embodiment of the presently disclosed subject matter and/or embodiments thereof, at least some of the monitoring devices are attached monitoring devices, attached to respective members.

In accordance with a second aspect of the presently disclosed subject matter, there is provided a method for estimating greenhouse gas emission in an environment housing an animal population, the method comprising: obtaining, by a processing circuitry, at least a subset of two or more records, each of the records (i) being associated with a respective member of members of the animal population, and (ii) including one or more monitored parameters of the respective member as monitored over time by at least one of a group of one or more monitoring devices configured to monitor parameters of the members of the animal population, wherein the subset is associated with a first group of given members of the members; determining, by the processing circuitry, one or more greenhouse gas emission affecting parameters based on the subset, wherein at least one greenhouse gas emission affecting parameter of the greenhouse gas emission affecting parameters is determined based on one or more of the monitored parameters that are monitored for at least two members of the first group of given members; estimating, by the processing circuitry, an amount of greenhouse gas emission in the environment utilizing the greenhouse gas emission affecting parameters; and determining, by the processing circuitry, one or more insurance parameters, for insuring at least one animal of the animal population, based on the estimated amount.

In one embodiment of the presently disclosed subject matter and/or embodiments thereof, the insurance parameters include one or more of: insurance price, insurance period, or obligations to meet in order to be eligible for insurance.

In one embodiment of the presently disclosed subject matter and/or embodiments thereof, the estimating is based on comparison of the greenhouse gas emission affecting parameters to a given baseline.

In one embodiment of the presently disclosed subject matter and/or embodiments thereof, the baseline is geographical location specific.

In one embodiment of the presently disclosed subject matter and/or embodiments thereof, the baseline is determined based on historical values of the greenhouse gas emission affecting parameters.

In one embodiment of the presently disclosed subject matter and/or embodiments thereof, the baseline is determined using greenhouse gas emission measurements acquired using greenhouse gas emission measuring equipment.

In one embodiment of the presently disclosed subject matter and/or embodiments thereof, the greenhouse gas emission measurements are acquired from the environment.

In one embodiment of the presently disclosed subject matter and/or embodiments thereof, the action is one or more of: (a) providing a user with an indication of the estimated amount of greenhouse gas emission, or (b) suggesting one or more greenhouse gas emission reduction actions to be taken on at least part of the animal population in order to reduce the greenhouse gas emission in the environment.

In one embodiment of the presently disclosed subject matter and/or embodiments thereof, the greenhouse gas emission reduction actions include one or more of: administering a treatment to the at least part of the animal population, changing a temperature of an environment of the at least part of the animal population, changing a feed of the at least part of the animal population, or changing a schedule of the at least part of the animal population.

In one embodiment of the presently disclosed subject matter and/or embodiments thereof, the greenhouse gas emission affecting parameters include one or more of the following: (a) a welfare score indicative of a health state of the first group of given members, (b) Heat Detection Rate (HDR) calculated for the first group of given members, (c) a conception rate calculated for the first group of given members, (d) a health score calculated for the first group of given members, (e) rumination consistency calculated for the first group of given members, or (f) rumination time heterogeneity calculated for the first group of given members.

In one embodiment of the presently disclosed subject matter and/or embodiments thereof, the method further comprises calculating, based on the subset of the records, for each given member of the first group of given members, at least one of: (a) an animal health score indicative of a health state of the respective member, (b) an animal natural living score, indicative of compliance of a behavior pattern of the respective member with a desired natural behavior pattern, or (c) an animal affectivity/happiness score, indicative of compliance of an affectivity/happiness measure of the respective member with a desired affectivity/happiness measure, the affectivity/happiness measure being determined based on one or more of the following monitored parameters, being affectivity/happiness parameters: (1) respiration level of the given member, (2) percentage of rumination time within a fourth time period of the given member, or (3) percentage of feeding time within a fifth time period of the given member; and wherein: the health score is calculated based on the animal health scores calculated for the first group of given members; and the welfare score is calculated based on the at least one of: (a) the health score, (b) the animal natural living scores calculated for the first group of given members, or (c) the animal affectivity/happiness scores calculated for the first group of given members.

In one embodiment of the presently disclosed subject matter and/or embodiments thereof, the monitored parameters include one or more of the following behavioral parameters: (a) percentage of movement time within a first time period of the given member, (b) percentage of feeding time within a second time period of the given member or (c) percentage of social behavior time within a third time period of the given member; and wherein the animal natural living score for the given member is determined based on the behavioral parameters.

In one embodiment of the presently disclosed subject matter and/or embodiments thereof, the natural living score for the given member is determined based on consistency of values of at least some of the behavioral parameters over time.

In one embodiment of the presently disclosed subject matter and/or embodiments thereof, the consistency of the values is measured against reference parameters.

In one embodiment of the presently disclosed subject matter and/or embodiments thereof, the reference parameters are measured from reference animals.

In one embodiment of the presently disclosed subject matter and/or embodiments thereof, the affectivity/happiness score for the given member is determined based on consistency of values of at least some of the affectivity/happiness parameters over time.

In one embodiment of the presently disclosed subject matter and/or embodiments thereof, the consistency of the values is measured against reference parameters.

In one embodiment of the presently disclosed subject matter and/or embodiments thereof, the reference parameters are measured from reference animals.

In one embodiment of the presently disclosed subject matter and/or embodiments thereof, the welfare score is calculated based on a variation between the affectivity/happiness scores of the first group of given members.

In one embodiment of the presently disclosed subject matter and/or embodiments thereof, the records also include one or more environmental parameters, indicative of the state of the environment of the respective member and wherein the determination of (a) the animal natural living score of the respective member or (b) of the animal affectivity/happiness score of the respective member, is also based on the environmental parameters.

In one embodiment of the presently disclosed subject matter and/or embodiments thereof, the monitoring devices include one or more of: an accelerometer, a temperature sensor, a location sensor, a pedometer, or a heart rate sensor.

In one embodiment of the presently disclosed subject matter and/or embodiments thereof, at least some of the records further include descriptive data associated with the respective member, wherein the descriptive data is not obtained from the monitoring devices.

In one embodiment of the presently disclosed subject matter and/or embodiments thereof, the descriptive data includes one or more of: age of the respective member, sex of the respective member, treatment history of the respective member, or genetic information associated with the respective member.

In one embodiment of the presently disclosed subject matter and/or embodiments thereof, at least some of the monitoring devices are attached monitoring devices, attached to respective members.

In accordance with a third aspect of the presently disclosed subject matter, there is provided a non-transitory computer readable storage medium having computer readable program code embodied therewith, the computer readable program code, executable by at least one processing circuitry of a computer to perform a method for estimating greenhouse gas emission in an environment housing an animal population, the method comprising: obtaining, by the processing circuitry, at least a subset of two or more records, each of the records (i) being associated with a respective member of members of the animal population, and (ii) including one or more monitored parameters of the respective member as monitored over time by at least one of a group of one or more monitoring devices configured to monitor parameters of the members of the animal population, wherein the subset is associated with a first group of given members of the members; determining, by the processing circuitry, one or more greenhouse gas emission affecting parameters based on the subset, wherein at least one greenhouse gas emission affecting parameter of the greenhouse gas emission affecting parameters is determined based on one or more of the monitored parameters that are monitored for at least two members of the first group of given members; estimating, by the processing circuitry, an amount of greenhouse gas emission in the environment utilizing the greenhouse gas emission affecting parameters; and determining, by the processing circuitry, one or more insurance parameters, for insuring at least one animal of the animal population, based on the estimated amount.

In accordance with a fourth aspect of the presently disclosed subject matter, there is provided an animal identity verification system, comprising a processing circuitry configured to: provide a data repository comprising one or more records, each of the records (a) including a unique animal identifier associated with a respective distinct animal of a plurality of animals, and (b) a Deoxyribonucleic acid (DNA) profile associated with the respective distinct animal; upon an animal identification requiring event being initiated for a given animal, obtain (a) a given animal identifier associated with the given animal, and (b) a given DNA profile extracted from a tissue sample of the given animal; retrieve the DNA profile associated with the given animal from the data repository, giving rise to an extracted DNA profile; compare the given DNA profile with the extracted DNA profile; and provide an authenticity indication of authenticity of the animal identity upon one or more authenticity requirements being met, the authenticity requirements including at least a first requirement for a match between the given DNA profile and the extracted DNA profile.

In one embodiment of the presently disclosed subject matter and/or embodiments thereof, the animal identification requiring event is one of the following: an insurance claim, a collateral claim, an animal sale, or a sale of a product generated by the animal or from the animal.

In one embodiment of the presently disclosed subject matter and/or embodiments thereof, the given animal identifier is obtained by reading an identification tag attached to the given animal.

In one embodiment of the presently disclosed subject matter and/or embodiments thereof, the identification tag is read by an electronic tag reader.

In one embodiment of the presently disclosed subject matter and/or embodiments thereof, the electronic tag reader is a tag reading wand.

In one embodiment of the presently disclosed subject matter and/or embodiments thereof, the authenticity requirements include a second requirement for validation of occurrence of the animal identification requiring event using readings obtained from a monitoring tag attached to the given animal, the monitoring tag configured to monitor parameters of the given animal.

In one embodiment of the presently disclosed subject matter and/or embodiments thereof, the parameters include at least one of: activity of the given animal or a body temperature of the given animal.

In one embodiment of the presently disclosed subject matter and/or embodiments thereof, the animal identification requiring event is death of the given animal and wherein the readings are indicative of the given animal's movements or body temperature, over time, before and after the insurance event.

In one embodiment of the presently disclosed subject matter and/or embodiments thereof, the DNA profile is generated using an extracted tissue sample extracted from the respective animal during tagging the respective animal with an identification tag, wherein the tagging results in the tissue sample being extracted from the respective animal.

In one embodiment of the presently disclosed subject matter and/or embodiments thereof, (a) each of the records further includes a reference image of the respective animal, (b) the obtaining includes obtaining a validation image of the given animal, (c) the retrieving includes retrieving the reference image associated with the given animal from the data repository, (d) the authenticity requirements include a third requirement for validating the identity of the given animal by matching the reference image with the validation image, (e) the identity of the given animal being validated upon the reference image matching the validation image.

In one embodiment of the presently disclosed subject matter and/or embodiments thereof, the reference image and the validation image are acquired using a user device.

In one embodiment of the presently disclosed subject matter and/or embodiments thereof, the reference image and the validation image are acquired from a substantially similar perspective of the given animal.

In one embodiment of the presently disclosed subject matter and/or embodiments thereof, the user device provides a user with instructions for capturing the validation image from the substantially similar perspective from which the reference image was acquired.

In one embodiment of the presently disclosed subject matter and/or embodiments thereof, (a) the data repository further includes, at least for the given animal, information enabling determination of an expected location of the given animal, being a geographical area in which the given animal is expected to be located, (b) the obtaining includes obtaining a validation location of the given animal, the validation location being determined subsequently to the animal identification requiring event, (c) the retrieving includes retrieving the expected location associated with the given animal from the data repository, (d) the authenticity requirements include a fourth requirement for validating the identity of the given animal by comparing the validation location with the expected location, (e) the identity of the given animal being validated upon the validation location being within the expected location, or at a pre-defined distance from the expected location.

In accordance with a fifth aspect of the presently disclosed subject matter, there is provided animal identity verification method comprising: providing a data repository comprising one or more records, each of the records (a) including a unique animal identifier associated with a respective distinct animal of a plurality of animals, and (b) a Deoxyribonucleic acid (DNA) profile associated with the respective distinct animal; upon an animal identification requiring event being initiated for a given animal, obtaining, by a processing circuitry, (a) a given animal identifier associated with the given animal, and (b) a given DNA profile extracted from a tissue sample of the given animal; retrieving, by the processing circuitry, the DNA profile associated with the given animal from the data repository, giving rise to an extracted DNA profile; comparing, by the processing circuitry, the given DNA profile with the extracted DNA profile; and providing, by the processing circuitry, an authenticity indication of authenticity of the animal identity upon one or more authenticity requirements being met, the authenticity requirements including at least a first requirement for a match between the given DNA profile and the extracted DNA profile.

In one embodiment of the presently disclosed subject matter and/or embodiments thereof, the animal identification requiring event is one of the following: an insurance claim, a collateral claim, an animal sale, or a sale of a product generated by the animal or from the animal.

In one embodiment of the presently disclosed subject matter and/or embodiments thereof, the given animal identifier is obtained by reading an identification tag attached to the given animal.

In one embodiment of the presently disclosed subject matter and/or embodiments thereof, the identification tag is read by an electronic tag reader.

In one embodiment of the presently disclosed subject matter and/or embodiments thereof, the electronic tag reader is a tag reading wand.

In one embodiment of the presently disclosed subject matter and/or embodiments thereof, the authenticity requirements include a second requirement for validation of occurrence of the animal identification requiring event using readings obtained from a monitoring tag attached to the given animal, the monitoring tag configured to monitor parameters of the given animal.

In one embodiment of the presently disclosed subject matter and/or embodiments thereof, the parameters include at least one of: activity of the given animal or a body temperature of the given animal.

In one embodiment of the presently disclosed subject matter and/or embodiments thereof, the animal identification requiring event is death of the given animal and wherein the readings are indicative of the given animal's movements or body temperature, over time, before and after the insurance event.

In one embodiment of the presently disclosed subject matter and/or embodiments thereof, the DNA profile is generated using an extracted tissue sample extracted from the respective animal during tagging the respective animal with an identification tag, wherein the tagging results in the tissue sample being extracted from the respective animal.

In one embodiment of the presently disclosed subject matter and/or embodiments thereof, (a) each of the records further includes a reference image of the respective animal, (b) the obtaining includes obtaining a validation image of the given animal, (c) the retrieving includes retrieving the reference image associated with the given animal from the data repository, (d) the authenticity requirements include a third requirement for validating the identity of the given animal by matching the reference image with the validation image, (e) the identity of the given animal being validated upon the reference image matching the validation image.

In one embodiment of the presently disclosed subject matter and/or embodiments thereof, the reference image and the validation image are acquired using a user device.

In one embodiment of the presently disclosed subject matter and/or embodiments thereof, the reference image and the validation image are acquired from a substantially similar perspective of the given animal.

In one embodiment of the presently disclosed subject matter and/or embodiments thereof, the user device provides a user with instructions for capturing the validation image from the substantially similar perspective from which the reference image was acquired.

In one embodiment of the presently disclosed subject matter and/or embodiments thereof, (a) the data repository further includes, at least for the given animal, information enabling determination of an expected location of the given animal, being a geographical area in which the given animal is expected to be located, (b) the obtaining includes obtaining a validation location of the given animal, the validation location being determined subsequently to the animal identification requiring event, (c) the retrieving includes retrieving the expected location associated with the given animal from the data repository, (d) the authenticity requirements include a fourth requirement for validating the identity of the given animal by comparing the validation location with the expected location, (e) the identity of the given animal being validated upon the validation location being within the expected location, or at a pre-defined distance from the expected location.

In accordance with a sixth aspect of the presently disclosed subject matter, there is provided a non-transitory computer readable storage medium having computer readable program code embodied therewith, the computer readable program code, executable by at least one processing circuitry of a computer to perform an animal identity verification method, the method comprising: providing a data repository comprising one or more records, each of the records (a) including a unique animal identifier associated with a respective distinct animal of a plurality of animals, and (b) a Deoxyribonucleic acid (DNA) profile associated with the respective distinct animal; upon an animal identification requiring event being initiated for a given animal, obtaining, by a processing circuitry, (a) a given animal identifier associated with the given animal, and (b) a given DNA profile extracted from a tissue sample of the given animal; retrieving, by the processing circuitry, the DNA profile associated with the given animal from the data repository, giving rise to an extracted DNA profile; comparing, by the processing circuitry, the given DNA profile with the extracted DNA profile; and providing, by the processing circuitry, an authenticity indication of authenticity of the animal identity upon one or more authenticity requirements being met, the authenticity requirements including at least a first requirement for a match between the given DNA profile and the extracted DNA profile.

### BRIEF DESCRIPTION OF THE DRAWINGS

In order to understand the presently disclosed subject matter and to see how it may be carried out in practice, the subject matter will now be described, by way of non-limiting examples only, with reference to the accompanying drawings, in which:

FIG. 1 is a schematic illustration of a process for verifying an identity of an animal, in accordance with the presently disclosed subject matter;

FIG. 2 is a block diagram schematically illustrating one example of an animal identity verification system, in accordance with the presently disclosed subject matter;

FIG. 3 is a flowchart illustrating one example of a sequence of operations carried out for gathering information required for verifying an identity of an animal, in accordance with the presently disclosed subject matter;

FIG. 4 is a flowchart illustrating one example of a sequence of operations carried out as part of verifying an identity of an animal, in accordance with the presently disclosed subject matter;

FIG. 5 is a flowchart illustrating one example of another sequence of operations carried out as part of verifying an identity of an animal, in accordance with the presently disclosed subject matter;

FIG. 6 is an exemplary screenshot of a display of a user device during an animal registration process as part of onboarding an animal, in accordance with the presently disclosed subject matter;

FIGS. 7a-7d are exemplary screenshots of dashboards, in accordance with the presently disclosed subject matter;

FIG. 8 is a block diagram schematically illustrating one example of an animal insurance parameters determination system, in accordance with the presently disclosed subject matter; and

FIG. 9 is a flowchart illustrating one example of a sequence of operations carried out for determining animal insurance parameters, in accordance with the presently disclosed subject matter.

### DETAILED DESCRIPTION

In the following detailed description, numerous specific details are set forth in order to provide a thorough understanding of the presently disclosed subject matter. However, it will be understood by those skilled in the art that the presently disclosed subject matter may be practiced without these specific details. In other instances, well-known methods, procedures, and components have not been described in detail so as not to obscure the presently disclosed subject matter.

In the drawings and descriptions set forth, identical reference numerals indicate those components that are common to different embodiments or configurations.

Unless specifically stated otherwise, as apparent from the following discussions, it is appreciated that throughout the specification discussions utilizing terms such as “obtaining”, “retrieving”, “comparing”, “providing” or the like, include action and/or processes of a computer that manipulate and/or transform data into other data, said data represented as physical quantities, e.g. such as electronic quantities, and/or said data representing the physical objects. The terms “computer”, “processor”, “processing circuitry” and “controller” should be expansively construed to cover any kind of electronic device with data processing capabilities, including, by way of non-limiting example, a personal desktop/laptop computer, a server, a computing system, a communication device, a smartphone, a tablet computer, a smart television, a processor (e.g. digital signal processor (DSP), a microcontroller, a field programmable gate array (FPGA), an application specific integrated circuit (ASIC), etc.), a group of multiple physical machines sharing performance of various tasks, virtual servers co-residing on a single physical machine, any other electronic computing device/s, and/or any combination thereof.

The operations in accordance with the teachings herein may be performed by a computer specially constructed for the desired purposes or by a general-purpose computer specially configured for the desired purpose by a computer program stored in a non-transitory computer readable storage medium. The term “non-transitory” is used herein to exclude transitory, propagating signals, but to otherwise include any volatile or non-volatile computer memory technology suitable to the application.

As used herein, the phrase “for example,” “such as”, “for instance” and variants thereof describe non-limiting embodiments of the presently disclosed subject matter. Reference in the specification to “one case”, “some cases”, “other cases” or variants thereof means that a particular feature, structure or characteristic described in connection with the embodiment(s) is included in at least one embodiment of the presently disclosed subject matter. Thus, the appearance of the phrase “one case”, “some cases”, “other cases” or variants thereof does not necessarily refer to the same embodiment(s).

It is appreciated that, unless specifically stated otherwise, certain features of the presently disclosed subject matter, which are, for clarity, described in the context of separate embodiments, may also be provided in combination in a single embodiment. Conversely, various features of the presently disclosed subject matter, which are, for brevity, described in the context of a single embodiment, may also be provided separately or in any suitable sub-combination.

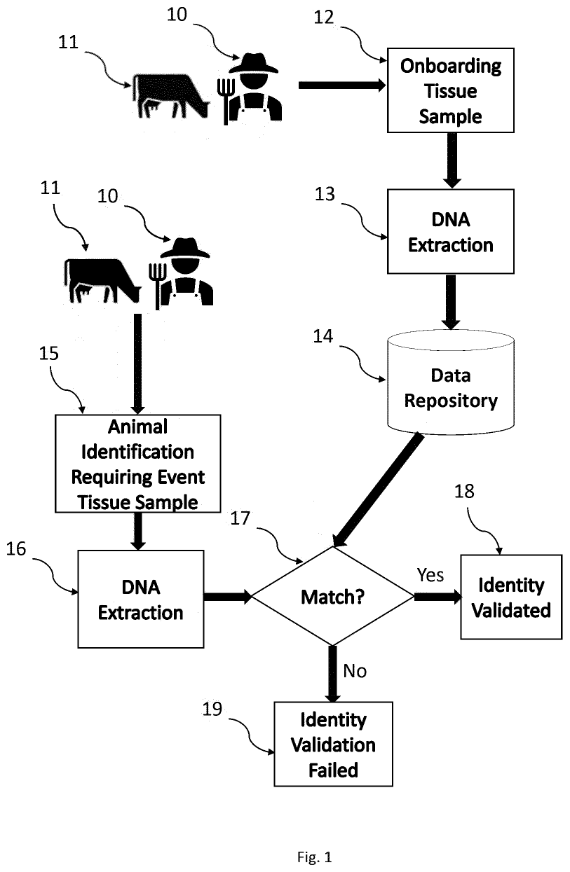

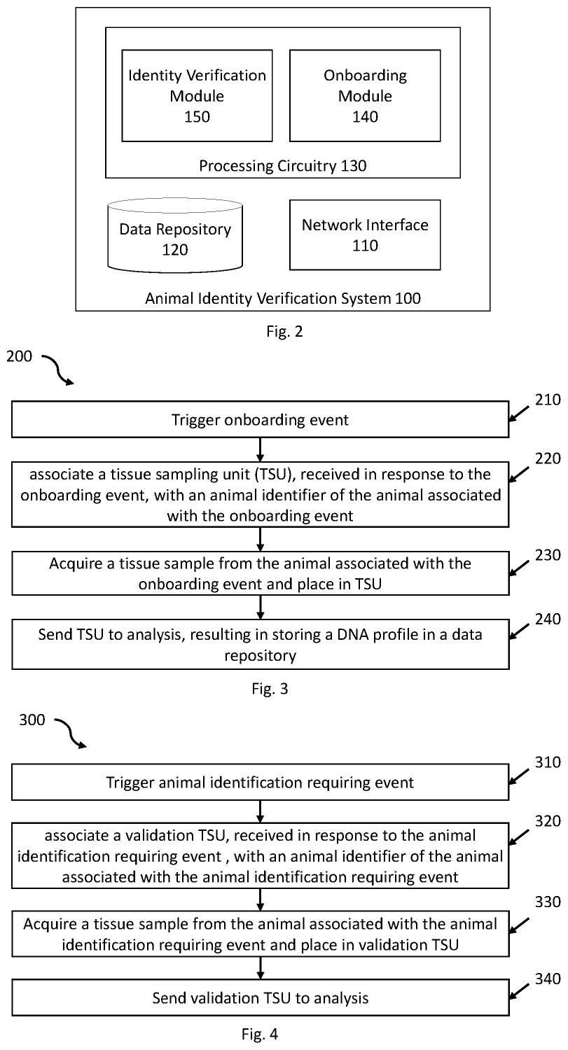

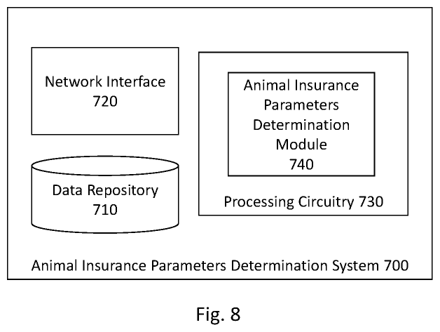

In embodiments of the presently disclosed subject matter, fewer, more and/or different stages than those shown in FIGS. 1, 3 to 5 and 9 may be executed. In embodiments of the presently disclosed subject matter one or more groups of stages illustrated in FIGS. 1, 3 to 5 and 9 may be executed simultaneously. FIGS. 2 and 8 illustrates a general schematic of the system architecture in accordance with an embodiment of the presently disclosed subject matter. Each module in FIGS. 2 and 8 can be made up of any combination of software, hardware and/or firmware that performs the functions as defined and explained herein. The modules in FIGS. 2 and 8 may be centralized in one location or dispersed over more than one location, as detailed herein. In other embodiments of the presently disclosed subject matter, the system may comprise fewer, more, and/or different modules than those shown in FIGS. 2 and 8.

Any reference in the specification to a method should be applied mutatis mutandis to a system capable of executing the method and should be applied mutatis mutandis to a non-transitory computer readable medium that stores instructions that once executed by a computer result in the execution of the method.

Any reference in the specification to a system should be applied mutatis mutandis to a method that may be executed by the system and should be applied mutatis mutandis to a non-transitory computer readable medium that stores instructions that may be executed by the system.

Any reference in the specification to a non-transitory computer readable medium should be applied mutatis mutandis to a system capable of executing the instructions stored in the non-transitory computer readable medium and should be applied mutatis mutandis to method that may be executed by a computer that reads the instructions stored in the non-transitory computer readable medium.

Bearing this in mind, attention is drawn to FIG. 1, which is a schematic illustration of a process for animal identity verification, in accordance with the presently disclosed subject matter.

In accordance with the example illustrated in the figure, a transaction requesting entity 10, such as a farmer, interested in carrying out a transaction associated with an animal 11 performs an onboarding process, during which the transaction requesting entity 10 obtains an onboarding tissue sample (block 12) (being a tissue sample acquired from the animal 11 during the onboarding process) from the animal 11. The transaction can be, for example, purchasing an insurance insuring the animal 11, obtaining a loan in which the animal 11 is used as collateral, selling the animal, or any other transaction which requires identification of the animal 11.

The onboarding tissue sample (obtained at block 12) undergoes Deoxyribonucleic acid (DNA) extraction (block 13) in order to extract a DNA profile biologically uniquely identifying the animal 11. The DNA profile obtained by the DNA extraction (block 13) is stored in a data repository (block 14). The DNA profile may optionally be associated with an additional animal identifier identifying the animal 11 (e.g. one or more of: an animal identification tag identifier of an animal identification tag attached to the animal 11, an animal monitoring tag identifier of an animal monitoring tag attached to the animal 11, a GPS location of the animal 11 (e.g. the animal's 11 location at the time of obtaining the tissue sample), a photo of the animal 11, etc.).

Upon an animal identification requiring event taking place (e.g. sale of the animal, or of a product generated by the animal or from the animal, collateral claim, insurance claim due to any event that results in compensation for the transaction requesting entity 10 according to an insurance policy associated with the animal 11), an animal identification process is initiated during which an animal identification requiring event tissue sample (being a tissue sample acquired from the animal 11 following occurrence of the insurance event) is acquired from the animal 11 (block 15), by the transaction requesting entity 10 or by another entity. The animal identification requiring event tissue sample (obtained at block 15) undergoes DNA extraction (block 16) in order to extract a DNA profile uniquely identifying the animal 11. Following extraction of the DNA profile, the DNA profile extracted during the animal identification process is compared with the DNA profile extracted during the onboarding process (which is extracted from the data repository for that purpose) in a DNA comparison stage (block 17). Upon the DNA profiles matching—the identity of the animal 11 is validated (block 18). Upon a mismatch between the DNA profiles the validation of the identity of the animal 11 the animal identity validation failed (block 19).

The above is a simplified explanation of the animal identity verification process, which is further explained herein, with reference to the following figures. However, it is to be noted that animal identity verification process can also be based on additional or other types of information, such as animal ID (obtained, for example by reading an animal ID tag attached to the animal), an image of the animal 11 (that can be compared to a reference image of the animal 11), a location of the animal 11 (obtained, for example by a GPS tracker that can acquire a reading indicative of the location of the animal 11, noting that such GPS tracker can be part of a tag attached to the animal 11 or it can be part of an external device such as a tag reader or a mobile device such as a smartphone), or some combination thereof. As one can appreciate, the more information used to verify the identity of the animal 11—the more accurate and less susceptible to fraud the animal identity verification is.

It is to be further noted that in some cases, the animal identity verification can be based on all, or some, of the above-mentioned additional information as an alternative to using the DNA profiling. In such cases, the animal identity verification is not based on a DNA profile of the animal.

Having described the general process of animal identity verification, attention is now drawn to FIG. 2. FIG. 2 is a block diagram schematically illustrating one example of an animal identity verification system, in accordance with the presently disclosed subject matter.

Animal identity verification system 100 comprises a network interface 110 (e.g. a network card, a WiFi client, a LiFi client, 3G/4G client, or any other component), enabling tracing system 100 to communicate over a network with user devices (e.g. devices operated by farmers), or other external systems, from which it obtains information of animal identifiers (e.g. animal identification tag identifiers of animal identification tags attached to animals and/or animal monitoring tag identifiers of animal monitoring tags attached to animals), data enabling determination of health-related parameters of animals (that can be obtained, for example, using animal monitoring tags attached to animals), images enabling identification of animals, data enabling non-biometric identification of animals (e.g. location data acquired by a Global Positioning System (GPS) tracker), etc.

Animal identity verification system 100 further comprises, or is otherwise associated with, a data repository 120 (e.g., a database, a storage system, a memory including Read Only Memory—ROM, Random Access Memory—RAM, or any other type of memory, etc.) configured to store data, optionally including, inter alia, information on animals, which can include, for each animal, one or more of: an animal identifier (which can be associated with an animal identification tag, such as an Electronic Identification (EID) tag or visual identification tag, attached to the animal), monitoring data (e.g., parameters of the animal that are monitored, optionally over time, using one or more monitoring devices, at least part of which can optionally be attached to the animal, and/or information extracted via analysis of such—or other types of—parameters, e.g. as further detailed herein), a DNA profile, one or more reference images (e.g. one or more images of the animal taken from one or more perspectives), historical health data (such as information of vaccinations and/or other medical treatments received by the animal, illness history, information of past vet checks, etc.), etc. Data repository 120 can be further configured to enable retrieval and/or update and/or deletion of the stored data. It is to be noted that in some cases, data repository 120 can be distributed, while the animal identity verification system 100 has access to the information stored thereon, e.g. via a wired or wireless network to which animal identity verification system 100 is able to connect (utilizing its network interface 110).

The monitoring data can include one or more of: location information (indicative of the geographical locations of the animal over time or at various points in time), body temperature readings, respiration type, respiration levels, rumination time, movement type, movement time, feeding time, social information, reproduction information, elimination behaviors, internal rumen environment parameters (which can be monitored, for example, using a rumen bolus), etc., all of which may be acquired over time, continuously, periodically, at various points in time, or at a single point in time.

As indicated herein, the monitoring data can also include information that is extracted via analysis of the parameters of the animal that are monitored, optionally over time, using one or more monitoring devices. Some examples include: indications of types and/or times of predicted, current and/or past illnesses of the animal; indications of times of predicted, current and/or past estrus of the animal; indications of failures to comply with regulatory requirements (e.g. missing vaccinations); information of improper transportation of the animal (e.g. failure to meet an arrival time requirement of arrival to a certain destination, failure to meet a departure time requirement of departure from a certain destination, failure to meet a stay time requirement of a minimal or maximal stay time at a certain destination, etc.); information of average milk production of the animal and/or of anomalies in the milk production; information indicative of events of the animal giving birth; information indicative of an estimated, or actual, age of the animal; the animal's health history; the animal's location and movement history; etc.

Some of the monitoring data (for example the raw data based on which additional monitoring data can be generated) can be obtained from sensors comprised within the monitoring device, such as: one or more accelerometers, a temperature sensor, a location sensor (e.g., a Global Positioning System (GPS) tracker comprised within the monitoring device attached to the member), a thermal sensor, a pedometer, an animal identification component (e.g. an Identification (ID) Tag), a heart rate sensor, a biosensor, or any other sensor that can be used to monitor one or more parameters of the animal monitored by the monitoring device. It is to be noted that the sensors can be external to the monitoring device and optionally not attached to a specific animal (e.g. cameras capturing images of the animal, locating means that can determine the location of the animal, etc.).

It is to be noted that in some cases, at least parts of the monitoring data can be measured over time. For example, rumination time, movement time, feeding time, socialism time, etc., can be the time from start to end of the respective activity. For example, if the animal started eating at 10:00 and finished eating at 10:30, the feeding time is 30 minutes. It is to be noted further that in some cases, at least parts of the monitoring data can be measured periodically, near continuously, or continuously, or in some combination (where some data is collected periodically and some data is collected continuously or near continuously). For example, body temperature, location, heart rate, etc. can be measured on a periodical basis (e.g., every minute, every 10 minutes, etc.), or continuously.

It is to be noted that whenever reference is made to continuously, near-continuously, real-time or near real time, the time intervals for obtaining a measurement can be milliseconds, seconds, minutes, hours or days, while noting that in comparison to current solutions in which human auditors are used, even time constants of days cannot be maintained, let alone time constants of hours, minutes, seconds or even less that are contemplated in accordance with the presently disclosed subject matter.

Animal identity verification system 100 further comprises a processing circuitry 130. Processing circuitry 130 can be one or more processing units (e.g. central processing units), microprocessors, microcontrollers (e.g. microcontroller units (MCUs)) or any other computing devices or modules, including multiple and/or parallel and/or distributed processing units, which are adapted to independently or cooperatively process data for controlling relevant animal identity verification system 100 resources and for enabling operations related to animal identity verification system's 100 resources.

Processing circuitry 130 can comprises one or more of the following modules: an onboarding module 140, and an identity verification module 150.

Onboarding module 140 is configured to perform at least part of an onboarding process for gathering information required for verification of an animal identity, as further detailed herein, inter alia with reference to FIG. 3.

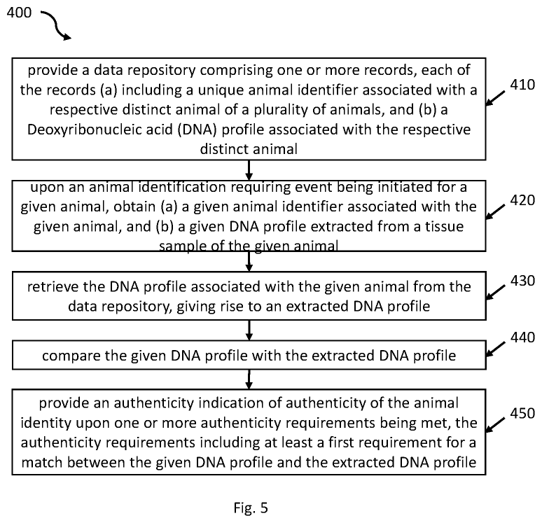

Identity verification module 150 is configured to perform at least part of an animal identity verification process upon occurrence of an animal identification requiring event, as further detailed herein, inter alia with reference to FIGS. 4 and 5.

Turning to FIG. 3, there is shown a flowchart illustrating one example of a sequence of operations carried out for gathering information required for verification of an animal identity, in accordance with the presently disclosed subject matter.

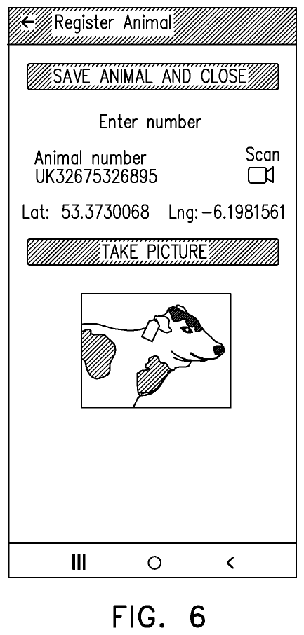

In accordance with the presently disclosed subject matter, the following onboarding process 200 can be performed for gathering information required for verification of an identity of an animal 11. The onboarding process 200 starts by triggering an onboarding event (block 210), optionally from an application installed on a mobile device (or any other computerized device having suitable capabilities) of the transaction requesting entity 10, or via a web-interface. As part of the onboarding event, information on the animal 11 is provided, including an identifier thereof (optionally obtained by reading an identification tag, such as an EID tag, attached to the animal 11), and optionally additional information, such as one or more images of the animal 11, information of the location of the animal 11, historical health data of the animal 11 (such as information of vaccinations and/or other medical treatments received by the animal 11, illness history of the animal 11, information of past vet checks performed on the animal 11, etc.), and/or any other information required in order to complete the transaction involving the animal 11. It is to be noted that the information provided by the transaction requesting entity 10, or parts thereof, can be acquired using the application installed on the mobile device (or any other computerized device having suitable capabilities) of the transaction requesting entity 10, or via a web-interface, as further detailed herein inter alia with reference to FIG. 6. Another source of information provided by the transaction requesting entity 10 is data repository 120 from which required data can be retrieved, optionally automatically.

In some cases, in response to the transaction requesting entity 10 requesting the transaction (e.g. requesting to purchase insurance for the animal 11, requesting a loan in which the animal 11 is used as collateral, requesting to sell the animal 11), a service provider providing animal identity verification services sends (directly, or indirectly) the transaction requesting entity 10 a Tissue Sampling Unit (TSU) into which a tissue sample collected from the animal 11 is to be inserted. In some cases, the TSU can be pre-associated with an animal identifier of the animal 11 involved in the desired transaction. In other cases, upon the transaction requesting entity 10 receiving the TSU, the transaction requesting entity 10 associates the TSU with an animal identifier of the animal 11 (block 220). Such association can be performed by the transaction requesting entity 10 inputting an identifier of the TSU and an identifier of the animal 11, optionally using a mobile phone (or any other computerized device having suitable capabilities) having a suitable application installed thereon, or via a web-interface. The identifier of the TSU and the identifier of the animal 11 can be manually inputted, or they can be acquired by scanning scannable codes attached to (or otherwise associated with) the TSU (e.g. a barcode, or any other scannable/readable code that can be scanned/read for example using a suitable code scanner/reader) and to the animal 11 (e.g. a barcode, a printed identifier, a sticker, a painted marking, an EID tag, or any other scannable/readable code that can be scanned/read for example using a suitable code scanner/reader).

It is to be noted that in some cases, the transaction requesting entity 10 may pre-possess a TSU that is not pre-associated with a specific animal, and the association between an identifier of the TSU and an identifier of the animal 11 can be performed by the transaction requesting entity 10 inputting an identifier of the TSU and an identifier of the animal 11 as explained hereinabove. In such cases, the TSU may not be sent to the transaction requesting entity 10 as part of the onboarding process 200, as the transaction requesting entity 10 already possesses the required TSU.

The transaction requesting entity 10 acquires a tissue sample from the animal 11 involved in the desired transaction and places in the TSU (block 230), which is then sent by the transaction requesting entity 10 to analysis resulting in storing a DNA profile of the animal 11 in a data repository 120 (e.g. using the onboarding module 140 that can associate the DNA profile with the animal identifier and optionally with additional information within the data repository 120) (block 240). The TSU can be sent by mail or by courier, optionally while being kept in appropriate conditions that ensure that it can be used in order to extract a DNA profile of the animal 11 therefrom.

It is to be noted that in some cases, the collected tissue sample itself can also be stored for future use. It is also contemplated that the tissue sample will be analyzed on-site (e.g. on the farm where the animal 11 is located) using suitable technology, and in such cases, there may not be a need to send the tissue sample to another location for analysis.

It is to be noted that the description of the onboarding process 200 provided herein assumes that the animal's 11 DNA profile was not previously extracted, e.g. for other purposes. In such cases, an existing DNA profile of the animal 11 can be used mutatis mutandis.

It is to be further noted that in some cases, the onboarding process can be performed (optionally from the application installed on the mobile device (or any other computerized device having suitable capabilities) of the transaction requesting entity 10, or via the web-interface) on a bulk of animals including the given animal 11, and in such cases the process is performed for each animal in the bulk. In some cases, the information that is acquired for each animal in the bulk of animals is determined based on a value of the animal, that can be determined, for example, by the animal's owner (e.g. a farmer), by a third party evaluator, by an insurer insuring the animal, or by another entity. For some animals whose worth is below a threshold, it may suffice to obtain an identifier thereof along with an image or location information thereof. For other animals whose worth is above the threshold but below a second threshold, it may suffice to obtain an identifier thereof along with an image and location information thereof. For animals whose worth is above the second threshold, it may be required to obtain an identifier thereof along with an image and location information thereof, and with a tissue sample thereof, as their high worth justifies collecting more expensive information, e.g. in order to prevent fraud that may have more severe consequences than had the animals been lower worth animals.

It is to be still further noted that in some cases, various parameters (e.g. cost, insurance coverage, etc.) of a service can be effected by the amount and/or type of information collected during an onboarding process of an animal, or group of animals. For example, if the service is insurance of the animal, or group of animals, the cost of the insurance can be reduced as the data collected on the animal, or group of animals, during the onboarding process is more complete or less susceptible to fraud (and vice versa—the less information collected on the animal or the more such information is susceptible to fraud—the higher the cost of the insurance can be). Alternatively, or additionally, for the same reasoning, the insurance coverage can be decreased when the data collected on the animal, or group of animals, during the onboarding process is less complete or more susceptible to fraud (and vice versa—the less information collected on the animal or the more such information is susceptible to fraud—the higher the cost of the insurance can be).

Looking at a specific example, the cost of the insurance of an animal for which a DNA profile enabling its identification was obtained can be lower than the cost of the insurance of an animal for which there is no DNA profile enabling its identification. Similarly, the insurance coverage of an insurance of an animal for which a DNA profile enabling its identification was obtained can be higher than the insurance coverage of an insurance of an animal for which there is no DNA profile enabling its identification.

Continuing the insurance example (while noting that the foregoing is relevant also for fields other than insurance), it should be noted that the cost of the insurance and/or the insurance coverage can also vary based on the monitoring data of an insured animal, or an insured group of animals. One type of monitoring data that can have an effect on the cost and/or coverage of an insurance is the location and/or location history of the animal (that can be tracked by various means, as detailed herein). For example, it is contemplated that the insurance cost can be higher if the animal is, or was, located at an area where disease is known to have been. Another type of monitoring data that can have an effect on the cost and/or coverage of an insurance is the Green House Gas (GHG) emission within a farm in which the animal or group of animals to be insured is/are located. It is contemplated that the insurance may be cheaper if the farm is producing less GHG. It is to be noted that the GHG emission levels of a farm can optionally be automatically determined based on analysis of the monitoring data. Yet another type of monitoring data that can have an effect on the cost and/or coverage of an insurance is the health and/or welfare of the animal or group of animals. The better the health and/or welfare of the animal or group of animals are—the lower the insurance cost can be and vice versa.

Animal health can be determined based on criteria of basic health and functioning of the animal or group of animals, including, specifically, freedom from disease and injury. In some embodiments, health, or a health score, can be determined based, at least in part, on identifying symptoms that can be associated with health-related issues (e.g., illness, injury, etc.). In some cases, the health score is determined based on finding statistically significant correlations between the identified symptoms and illnesses or injuries. In some cases, the strengths of such correlations between the symptoms that are indicative of illness and/or injuries is also used in order to determine the health score.

Those symptoms can be identified by monitoring rumination and/or energy levels of an individual animal, or group of animals (based on ownership, location, etc.). Additionally, or alternatively, the symptoms can be identified by based, at least in part, on monitoring visual indicators associated with health problems and/or monitoring breathing patterns of an individual animal, or group of animals.

Energy level can be measured, for example, by detecting movements of an animal over a given time period (e.g., a day) and deducing the amount of energy required from the animal to perform these movements (optionally based on specific characteristics of the animal). The movements can be detected by monitoring an acceleration signal obtained for example from an accelerometer comprised within a monitoring device attached to the animal.

Rumination can be measured, for example, by detecting ruminating activity of an animal over a given time period (e.g., a day). This can be achieved by analyzing acceleration signal obtained for example from an accelerometer comprised within a monitoring device attached to the animal and detecting signals that correlate to reference signals that are known to be associated with rumination activity.

Breathing patterns can be determined, for example, by detecting various breathing patterns of an animal over a given time period (e.g., a day). This can be achieved by analyzing acceleration signal obtained for example from an accelerometer comprised within a monitoring device attached to the animal and detecting signals that correlate to reference signals that are known to be associated with specific breathing patterns. This can enable determining whether the animal demonstrates suspicious behavior (e.g., if it is breathing heavily more than usual and/or over a predetermined threshold, etc.).

Visual indicators associated with health problems can be identified using image analysis of images and/or videos of the animal, or group of animals. For example, scratching or irritation that are correlated with morbidity symptoms can be identified using image and/or video analysis.

It is to be noted that these are mere examples for determination of symptoms that can be associated with health-related issues, and additional and/or alternative data can be used to determine the above indicated symptoms, or other symptoms that can be associated with health-related issues.

The health score of animals within a group of animals can be used for calculating a health index indicative of the overall health level of the animals in the group. In some cases, the health index can be used for identifying morbidity levels in the group. For example, a health index showing that more than a threshold (e.g., around 5%) of the animals are having health scores below a threshold can be an indication of a developing health problem within that animal population. A health index showing that more than a second threshold (e.g., around 10%) of the animals are having health scores below a threshold can be an indication of a prevailing health problem. A health index showing that more than a third threshold (e.g., around 20%) of the animals are having health scores below a threshold can be indicative of a pandemic.

It is to be noted that analysis of the health scores can enable identification of given animals of the group of animals that have a certain health issue (e.g., illness, lameness, estrus, etc.). In some cases, analysis of the health index (generated based on the health scores of the animals in the group) can enable, at the population level, identifying whether the overall health level of the animal in the group is above an acceptable predetermined health threshold.

It is to be noted that historical health data of the animal 11 (such as information of vaccinations and/or other medical treatments received by the animal 11, illness history of the animal 11, information of past vet checks performed on the animal 11, etc.), can also be used in determining its health which can have an effect on the transaction costs. The health data of individual animals can be used also for determining a health score for a group of animals, e.g. as part of the process detailed herein.

Turning to animal welfare, it can be determined for a specific animal or for a group of animals using one or more animal welfare Key Performance Indicators (KPIs). The animal welfare KPIs can include a health score (that can be determined as detailed herein), an affectivity/happiness score and/or a natural living score.

The calculation of affectivity/happiness score for a given animal can be based on a state the animal is in, for example: pain, distress, frustration, pleasure, etc. Affectivity/happiness score can reflect how the animal is affected by its environment and its experiences (is it positively affected by its environment or negatively affected by its environment). In some cases, the calculation of affectivity/happiness score for an animal is based on level of suffering of the animal, so that the more suffering is identified—the lower its affectivity/happiness score is. The state of the animal, or its level of suffering, can be determined, for example, based on changes in its breathing patterns, its rumination activity, its eating activity, or some combination thereof. For example: measuring the variability of rumination and eating times, and levels of heavy breathing between members of a group of animals, optionally on a daily basis, can provide a basis for the affectivity/happiness score.

The calculation of the natural living score can be based on analysis of behaviors indicative of the ability of the animal to live a reasonably natural life by carrying out natural behavior and having access to natural elements in its environment. These specific behaviors can include: eating activity, grazing activity, activity level (e.g., the quantity and intensity of movement of the given animal), walking activity, etc. These behaviors can be used to verify that animals have, for example, enough time and opportunity to eat, express normal high-levels of activity, are not forced to walk to much, or are restricted from appropriate amounts of movement/walking. A non-limiting example related to the natural living behavior for dairy cows is in a dairy farm wherein calves may be regularly separated from their mothers within the first day after birth, and are fed milk from a bucket, usually twice per day. With such infrequent meals the total milk intake is limited so that the calf does not receive too much milk at one time. By contrast, under natural conditions, cows stay fairly close to the calves for the first two weeks, and the calf will feed many times per day in smaller meals. Adjusting the feeding systems to correspond more closely to the animals' natural behavior (staying close to the mother and feeding often but in relatively smaller amounts) will result in a higher natural living score for that calf. An additional example related to the natural living behavior is the time spent by a grazing animal in the pasture looking for food that can suggest a need for fencing reallocation.

It is to be noted, with reference to FIG. 3, that some of the blocks can be integrated into a consolidated block or can be broken down to a few blocks and/or other blocks may be added. Furthermore, in some cases, the blocks can be performed in a different order than described herein (e.g. block 230 can be performed before block 220). It is to be further noted that some of the blocks are optional (e.g. block 220).

Attention is now drawn to FIG. 4, a flowchart illustrating one example of a sequence of operations carried out as part of verifying an identity of an animal, in accordance with the presently disclosed subject matter.

In accordance with the presently disclosed subject matter, the following animal identity validation data collection process 300 can be performed as part of a process for verifying an identity of an animal. The animal identity validation data collection process 300 can start by an animal identity verification requiring entity (also referred to interchangeably herein as “identity verifier”), such as the transaction requesting entity 10, or any other entity that is interested in verifying the identity of the animal 11, triggering an animal identification requiring event (block 310). For example, the identity verifier can be the transaction requesting entity 10 (e.g. a farmer) that has insured the animal 11, whereas the animal identification requiring event is an insurance claim, in which the identity of the insured animal needs to be verified in order to collect the compensation to which the farmer is entitled when the insurance triggering event occurs (e.g. animal death, animal illness, etc.). Alternatively, the identity verifier can be a legal entity (such as a person or a company) interested in purchasing the animal 11 or in collecting the animal 11 when the animal's 11 owner fails to repay a loan and the animal 11 is used as collateral to such loan.

In some cases, in response to the triggering of the animal identification requiring event, a service provider providing animal identity verification services sends (directly, or indirectly) the identity verifier a validation TSU into which a new tissue sample collected from the animal 11 is to be inserted for validating the identity of the animal 11. In some cases, the validation TSU can be pre-associated with an animal identifier of the animal 11 involved in the desired transaction. In other cases, upon the identity verifier receiving the validation TSU, the identity verifier associates the validation TSU with an animal identifier of the animal 11 (block 320). Such association can be performed by the identity verifier inputting an identifier of the validation TSU and an identifier of the animal 11, optionally using a mobile phone (or any other computerized device having suitable capabilities) having a suitable application installed thereon, or via a web-interface. The identifier of the validation TSU and the identifier of the animal 11 can be manually inputted, or they can be acquired by scanning scannable codes attached to (or otherwise associated with) the validation TSU (e.g. a barcode, or any other scannable/readable code that can be scanned/read for example using a suitable code scanner/reader) and to the animal 11 (e.g. a barcode, a printed identifier, a sticker, a painted marking, an EID tag, or any other scannable/readable code that can be scanned/read for example using a suitable code scanner/reader).

It is to be noted that in some cases, the identity verifier may pre-possess the validation TSU that is not pre-associated with a specific animal. The association between an identifier of the validation TSU and an identifier of the animal 11 can be performed by the identity verifier inputting an identifier of the TSU and an identifier of the animal 11 as explained hereinabove. In such cases, the validation TSU may not be sent to the identity verifier as part of the animal identity validation data collection process 300, as the identity verifier already possesses the required validation TSU. In order to exemplify, the identity verifier can receive at a certain point in time a plurality of validation TSUs that are not pre-associated with specific animals. In such case, the identity verifier can use the validation TSUs when required in a process that includes associating a validation TSU being used with a specific animal from which a tissue sample is collected (using the validation TSU identifier and an identifier of such animal).

The identity verifier acquires a tissue sample from the animal 11 involved in the desired transaction and places in TSU (block 330), which is then sent by the identity verifier to analysis resulting in extraction of a DNA profile of the animal 11 that can be used for comparison with the previously extracted DNA profile, extracted during the onboarding process 200, as further detailed herein, inter alia with reference to FIG. 5 (block 340). The TSU can be sent by mail or by courier, while being kept in appropriate conditions that ensure that it can be used in order to extract a DNA profile of the animal 11 therefrom.

As indicated herein, it is to be noted that in some cases, the collected tissue sample itself can be stored for future use. It is also contemplated that the tissue sample will be analyzed on-site (e.g. on the farm where the animal 11 is located) using suitable technology, and in such cases, there may not be a need to send the tissue sample to another location for analysis.

It is to be noted, with reference to FIG. 4, that some of the blocks can be integrated into a consolidated block or can be broken down to a few blocks and/or other blocks may be added. Furthermore, in some cases, the blocks can be performed in a different order than described herein (e.g. block 230 can be performed before block 220). It is to be further noted that some of the blocks are optional (e.g. block 220). It should be also noted that whilst the flow diagram is described also with reference to the system elements that realizes them, this is by no means binding, and the blocks can be performed by elements other than those described herein.

Attention is drawn to FIG. 5, a flowchart illustrating one example of another sequence of operations carried out as part of verifying an identity of an animal, in accordance with the presently disclosed subject matter.

In accordance with the presently disclosed subject matter, the following animal identity verification process 400 can be performed, e.g. utilizing identity verification module 150.

For this purpose, the animal identification system 100 can be configured to provide a data repository 120 comprising one or more records, each of the records (a) including a unique animal identifier associated with a respective distinct animal of a plurality of animals, and (b) a DNA profile associated with the respective distinct animal (block 410). New records can be inserted into the data repository 120 in as part of the onboarding process 200 described herein, or in other manners (e.g. manually, automatically from another system having such information, etc.).

It is to be noted that all, or some, of the DNA profiles stored in the data repository 120 are generated using an extracted tissue sample extracted from the respective animals during tagging the respective animals with an identification tag, wherein the tagging results in the tissue sample being extracted from the respective animals.

Upon an animal identification requiring event being initiated for a given animal 11, the animal identification system 100 can be configured to obtain (a) a given animal identifier associated with the given animal 11, and (b) a given DNA profile extracted from a tissue sample of the given animal 11 (block 420).

The animal identification requiring event can be, for example, sale of the given animal 11 (e.g. where the purchaser of the given animal 11 wishes to verify that it is indeed a specific animal whose purchase was previously negotiated), sale of a product generated by the animal or from the animal (such as sale of milk or beef, in which the purchaser wishes to verify the origin of the product, e.g. what cow/farm/type of cow/country did the product originate from), collateral claim in which the collateral is the given animal 11 (e.g. where the animal owner fails to repay a loan for which the given animal 11 is used as collateral and wants to verify that the animal that is collected is indeed the one used as collateral), insurance claim due to any event that results in compensation for the transaction requesting entity 10 according to an insurance policy associated with the given animal 11 (e.g. where the insurer wishes to verify that the insurance event actually involved the given animal 11 and not another animal).

The given animal identifier can be obtained from a device other than the animal identification system 100, which can obtain it for example, by reading an identification tag attached to the given animal 11 using an electronic tag reader (such as a tag reading wand or any other electronic tag reader having capabilities of reading the given animal 11 identifier from an ID tag attached thereto). Such other device can provide the given animal identifier to the animal identification system 100 (e.g. by sending it over a communication network, directly, or via an intermediary device such as a mobile phone of the animal's owner). It is to be noted that the given animal identifier can be obtained by other means, including by manually inputting an identifier of the given animal 11, by determining an identity of the given animal 11 using image processing, or by any other means that enable obtaining the given animal identifier. The given DNA profile can be obtained by extracting it from a tissue sample as detailed with respect to the animal identity validation data collection process 300.

Animal identification system 100 can be further configured to retrieve the DNA profile associated with the given animal 11 (based on the given animal identifier obtained at block 420) from the data repository 120, giving rise to an extracted DNA profile (block 430).

Animal identification system 100 can then compare the given DNA profile with the extracted DNA profile (block 440), and provide an authenticity indication of authenticity of the given animal's 11 identity upon one or more authenticity requirements being met (block 450). The authenticity requirements can include at least a first requirement for a match between the given DNA profile and the extracted DNA profile, however additional authenticity requirements may exist, which complement the DNA profile matching. It is to be noted that in some cases, the additional authenticity requirements may actually be alternatives to the DNA profile matching, which in such cases is not performed.

The authenticity requirements may include, for example, a requirement for validation of occurrence of the animal identification requiring event using readings obtained from a monitoring tag attached to the given animal 11. A monitoring tag is configured to monitor parameters of the given animal 11 as described herein, including at least one of: activity of the given animal 11 or a body temperature of the given animal 11. As indicated herein, the monitoring data can also include information that is extracted via analysis of the parameters of the animal that are monitored (such as the activity of the given animal and/or the body temperature of the given animal), optionally over time. Such information can also be used as part of the animal identity verification process 400.

In a specific example, the animal identification requiring event is death of the given animal 11 and the readings are indicative of the given animal's 11 movements or body temperature, over time, before and after the animal identification requiring event. For example, when the given animal 11 is insured and compensation is due in case the given animal 11 dies, the insurer may wish to verify that the given animal 11 actually died. The readings obtained from the monitoring tag may enable validating the death of the given animal 11, which is expected to have a certain body temperature range before its death and a lower body temperature a certain time after its death (e.g. an hour after its death or even less). Similarly, the given animal 11 is expected to move before its death and stop moving after its death.

Another option for validating death of an animal is by analyzing (a) a monitored temperature time series of monitored temperature values that are indicative of a temperature of the animal over a given time period (e.g. 2-5 days) prior to the animal's death, and/or (b) a monitored acceleration time series of monitored acceleration values that are indicative of an acceleration of the animal over the given time period prior to the animal's death. For example, the analysis can be made using a machine learning model trained using historical readings obtained from animals (both animals that died within a given time period (e.g. 2-5 days) from the first reading and animals that did not die during such time period).

Returning to another example of an animal identification requiring event, the animal identification requiring event can be purchase of an animal. In such case, the identity of the purchased animal can be verified also using the monitoring information. The monitoring information can also be used in order to determine a price for the purchased animal, e.g. based on its current and historical health, its average milk yield (optionally during a certain time window such as 12 months prior to its purchase), its time between estruses, its eating and/or drinking habits, etc. In this respect it is to be further noted that analysis of the animal's DNA (using the tissue sample collected from the animal) can also enable determining a breed of the animal (e.g. is it an Aberdeen Angus, or another breed), which can also impact the animal's pricing.

The authenticity requirements may additionally or alternatively include a requirement for validating the identity of the given animal 11 by matching a reference image thereof (acquired prior to the animal identification requiring event) with a validation image thereof (acquired after the animal identification requiring event). In such cases, each, or at least some, of the records further includes a reference image of the respective animal (acquired prior to the animal identification requiring event); the obtaining of block 420 includes obtaining a validation image of the given animal 11 (acquired after the animal identification requiring event); the retrieving of block 430 includes retrieving the reference image associated with the given animal 11 from the data repository; and the identity of the given animal 11 is validated upon the reference image matching the validation image (upon all other authenticity requirements being met).

It is to be noted that in some cases the reference image and the validation image are acquired using a user device, such as a mobile phone. It is to be further noted that in some cases, in order for the matching to be successful, the reference image and the validation image are required to be acquired from a substantially similar perspective of the given animal. In order to accomplish this, in some cases, the given animal 11 can be held in a certain posture in order to ensure that the reference image/s and the validation image are captured from substantially the same distance and/or perspective with respect to the given animal 11.

Alternatively, or additionally, the user device used to acquire the validation image can be configured to provide the user taking the validation image with instructions for capturing the validation image from a substantially similar perspective from which the reference image was acquired. This can be performed by analyzing the reference image and providing the user with instructions for capturing the validation image from substantially the same distance and/or perspective.

In some cases, the reference image and the validation image are required to include an EID tag attached to the animal, or any other identifier that is attached to the given animal 11 and/or marked on the given animal 11.

The authenticity requirements may additionally or alternatively include a requirement for validating the identity of the given animal by comparing a validation location thereof (acquired after the animal identification requiring event) with an expected location.

In such cases, the data repository 120 further includes, at least for the given animal 11, information enabling determination of an expected location of the given animal 11, being a geographical area in which the given animal 11 is expected to be located (this can be determined based on past locations of the animal, that can be determined, for example, using: (a) readings obtained from a GPS tracker attached to the given animal 11, optionally within an identification tag (an EID) or a monitoring tag attached thereto, (b) readings of an EID tag by tag readers that are located at known locations or at locations that can be determined using GPS within the tag readers, or within a device communicatively connected to the tag readers (e.g. a mobile phone that communicates with the tag reader), (c) using Ultra Wide Band tracking technologies to track the location of an EID tag within a certain area, etc.); the obtaining of block 420 includes obtaining a validation location of the given animal 11, the validation location being determined subsequently to the animal identification requiring event; the retrieving of block 430 includes retrieving the expected location associated with the given animal 11 from the data repository; and the identity of the given animal is validated upon the validation location being within the expected location, or at a pre-defined distance from the expected location (upon all other authenticity requirements being met). It is to be noted that the validation location can be obtained by reading a GPS location of the given animal 11 from a GPS tracker attached to the given animal 11 (noting that the GPS tracker can be comprised within a tag attached to the given animal 11 such as an EID tag or a monitoring tag) or from a GPS tracker of a mobile phone of a person in vicinity to the given animal 11 (e.g. a farmer), or from any other GPS tracker that can determine a GPS location of the given animal 11.

It is to be noted, with reference to FIG. 5, that some of the blocks can be integrated into a consolidated block or can be broken down to a few blocks and/or other blocks may be added. It should be also noted that whilst the flow diagram is described also with reference to the system elements that realizes them, this is by no means binding, and the blocks can be performed by elements other than those described herein.

Attention is now drawn to FIG. 6, showing an exemplary screenshot of a display of a user device during an animal registration process as part of onboarding an animal, in accordance with the presently disclosed subject matter.

As can be appreciated, the user interface shown on the display of the user device includes an animal identifier section, into which the animal identifier is inputted (e.g. manually or by reading an identification tag attached to the animal 11).

The user interface further includes a section into which the current location of the animal is to be inputted. The current location of the animal can be determined by a GPS transceiver of the user device itself, or it can be retrieved from a GPS tracker attached to the animal (e.g. comprised within an ID tag or a monitoring tag attached to the animal). It is to be noted that this information can be later used as the expected location, or as a baseline for determining the expected location that may be used as part of the authenticity requirements as detailed herein, inter alia with reference to FIG. 5.

The user interface further includes a section for inputting an image of the animal. The image can be acquired in real time using a camera of the user device, or it can be acquired by an external camera and provided to the user device via a communication network or in any other manner. It is to be noted that in some cases, the image of the animal can be obtained from other sources that can optionally be accessible via an Internet connection. The image of the animal can later be used as the reference image that may be used as part of the authenticity requirements as detailed herein, inter alia with reference to FIG. 5.

It is to be noted that the user interface shown in FIG. 6 is merely an example and the user interface can be different than the one shown. For example, the user interface can include other sections than the ones shown herein (e.g. a section for inputting an identifier of a TSU used for collecting the onboarding tissue sample), and/or some of the sections shown herein may be omitted.

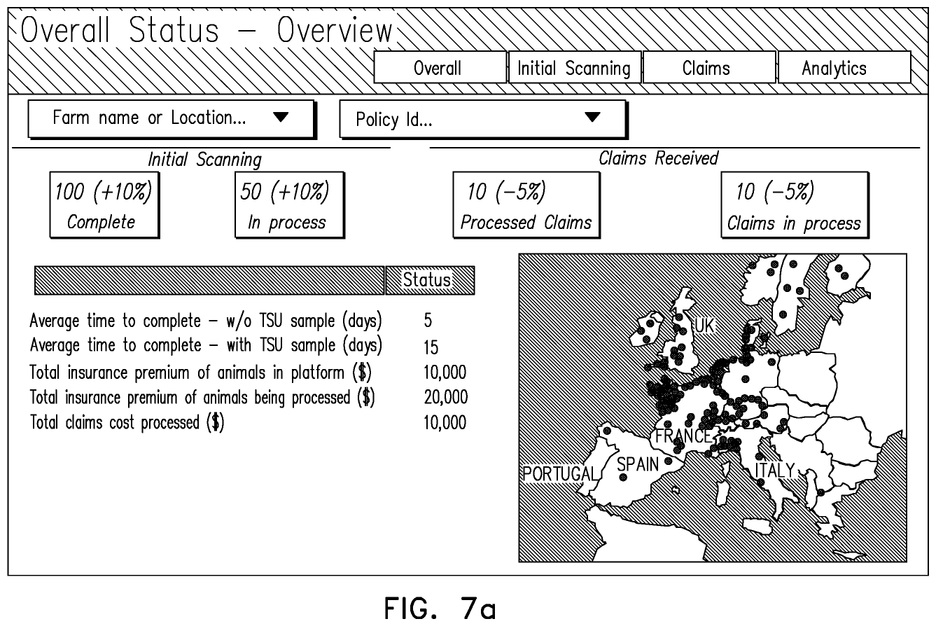

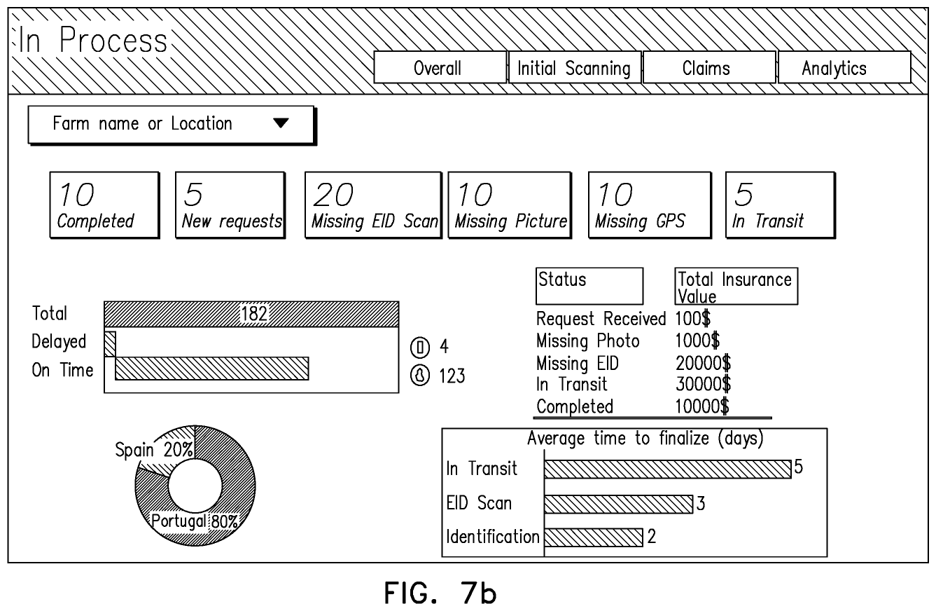

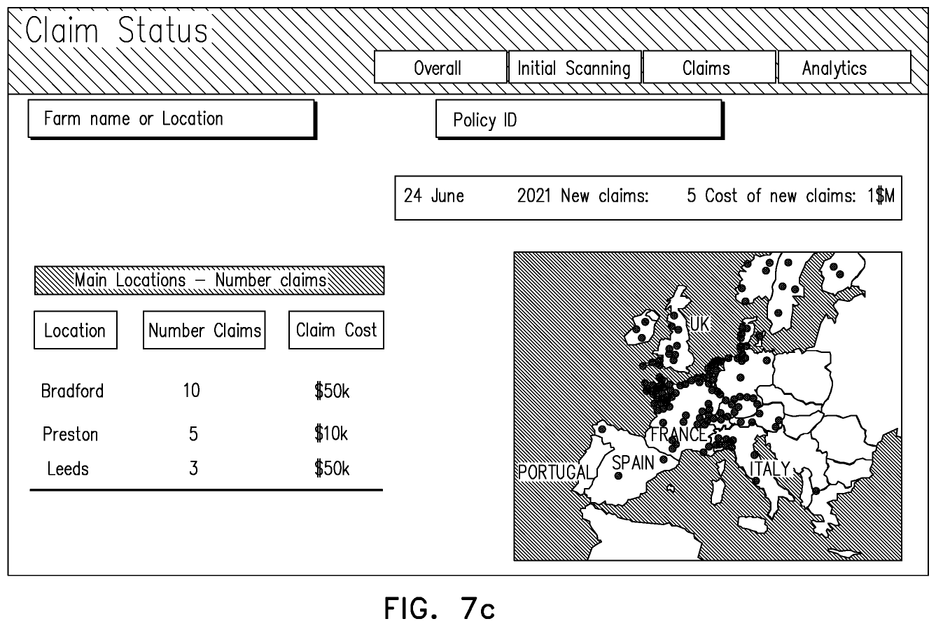

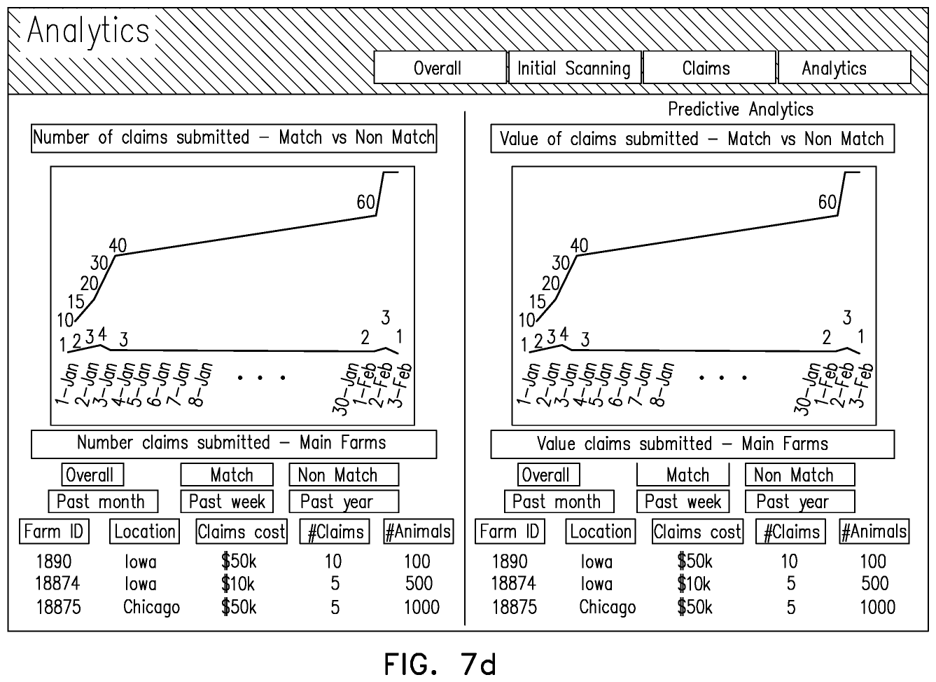

Turning to FIGS. 7a-7d, there are shown exemplary screenshots of dashboards, in accordance with the presently disclosed subject matter. The dashboards can be used by various stakeholders, such as the animals' owner, an insurer, a lender, a sales house, or someone on their behalf, or any other stakeholder. The dashboard shown in FIG. 7a shows, on the left-hand side, the number of animals that have completed the onboarding stage and the number of animals that haven't completed the onboarding stage. On the right-hand side, there is shown the number of claims with respect to insured animals that have been processed and the number of claims with respect to insured animals that are still in process (while noting that the dashboards that are provided in the examples in FIGS. 7a-7d refer to animal insurance, however adjustment can be made for other types of transactions, mutatis mutandis). In addition, various statistical information can also be provided as shown on the lower left-hand side of the dashboard (e.g. average time to complete onboarding with tissue sampling, average time to complete onboarding without tissue sampling, total insurance premium of insured animals that have completed the onboarding, total insurance premium of animals that haven't completed the onboarding, total claims cost of processed claims, etc.). The information that is shown can be filtered based on location (e.g. country, district, etc.), farm, multiple farms having a common ownership, specific insurance policy identifier, etc.

The dashboard shown in FIG. 7b shows, for a specific location or farm, how many animals have completed the onboarding, how many animals need to start the onboarding process, how many animals do not have an EID scan and/or a reference image and/or a location reading, and how many animals are in transit to, or from, the specific location or farm. In addition, there is shown a table including information on the total insurance value of the animals based on the processing stage of the various animals within the given location or farm. There is also shown a graph indicating the average number of days required to complete each stage of the onboarding.

The dashboard shown in FIG. 7c shows, for specific locations or farms, how many claims have been made with respect to insured animals, and the cost of the claims. The dashboard shown in FIG. 7d shows, for a specific location or farm, how many claims have been made in which the animal's identity has been verified, how many claims have been made in which the animal's identify verification resulted in the verification failing (i.e., indicating that the claim may be fraudulent), the values of the claims, etc.

It is to be noted that the dashboards shown in FIGS. 7a-7d are non-limiting examples only and the user interface and/or the information shown therein, can be different than the one shown. For example, the user interface can include other sections than the ones shown herein, and/or some of the sections shown herein may be omitted.

Attention is now drawn to FIG. 8. FIG. 8 is block diagram schematically illustrating one example of an animal insurance parameters determination system, in accordance with the presently disclosed subject matter. Animal insurance parameters determination system can be configured to estimate greenhouse gas emission in an environment housing an animal population and use it in order to determine animal insurance parameters, as further detailed herein.

Animal insurance parameters determination system 700 comprises a network interface 720 (e.g., a network card, a WiFi client, a LiFi client, 3G/4G client, or any other component), enabling animal insurance parameters determination system 700 to communicate over a network with external systems from which it obtains monitored parameters of members of an animal population and/or descriptive data associated with the members. The external systems can be monitoring devices configured to monitor parameters of the members, or any other intermediate system(s) that obtain the information about the members from the monitoring devices (e.g., computerized systems that manage at least part of the members of the animal population).

Animal insurance parameters determination system 700 further comprises, or is otherwise associated with, a data repository 710 (e.g., a database, a storage system, a memory including Read Only Memory—ROM, Random Access Memory—RAM, or any other type of memory, etc.) configured to store data, optionally including, inter alia, animal information records. Each information record is associated with a distinct member of the animal population and can include descriptive data of the member (e.g., age of the distinct member, sex of the distinct member, treatment history of the distinct member, genetic information associated with the distinct member, etc.) and monitoring data and/or monitored parameters of the distinct member as monitored by the monitoring devices over time (e.g., health parameters, behavioral parameters, affectivity/happiness parameters, etc.). Data repository 710 can be further configured to enable retrieval and/or update and/or deletion of the stored data. It is to be noted that in some cases, data repository 710 can be distributed, while the animal insurance parameters determination system 700 has access to the information stored thereon, e.g., via a wired or wireless network to which animal insurance parameters determination system 700 is able to connect (utilizing its network interface 720).

Animal insurance parameters determination system 700 further comprises a processing circuitry 730. Processing circuitry 730 can be one or more processing units (e.g., central processing units), microprocessors, microcontrollers (e.g., microcontroller units (MCUs)) or any other computing devices or modules, including multiple and/or parallel and/or distributed processing units, which are adapted to independently or cooperatively process data for controlling relevant animal insurance parameters determination system 700 resources and for enabling operations related to animal insurance parameters determination system's 700 resources.

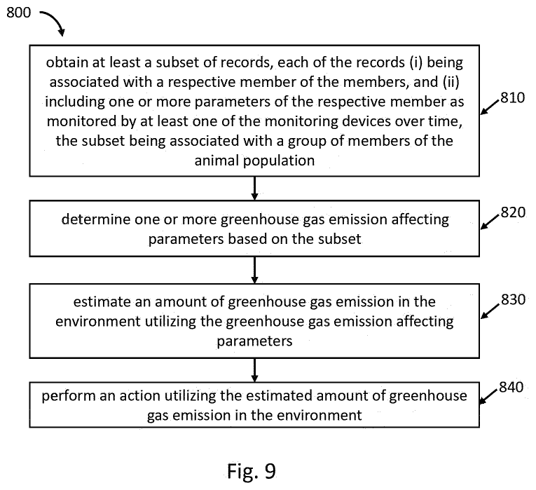

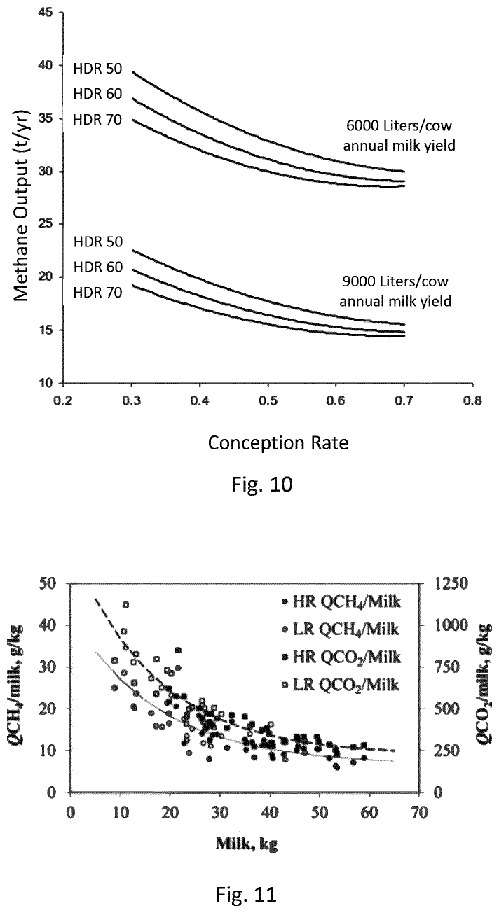

Processing circuitry 730 can comprise an animal insurance parameters determination module 740. Animal insurance parameters determination module 740, can be configured to determine animal insurance parameters, as further detailed herein, inter alia with reference to FIG. 9.

Turning to FIG. 9, there is shown a flowchart illustrating one example of a sequence of operations carried out for determining animal insurance parameters, in accordance with the presently disclosed subject matter.

In accordance with the presently disclosed subject matter, animal insurance parameters determination system 700 can be configured to perform an animal insurance parameters determination process 800 (optionally in real-time or near-real time and optionally in a continuous or near-continuous manner), e.g., using animal insurance parameters determination module 740.

As indicated herein, data gathered from the monitoring devices is stored in records within a data repository 710. Each record is associated with a respective member of the animal population and holds descriptive data (e.g., age of the respective member, sex of the respective member, treatment history of the respective member, genetic information associated with the respective member, etc.) and monitoring data, e.g., parameters of the animal (for example: temperature, movement time, feeding time, social behavior time, respiration levels, rumination time, etc.) obtained from the monitoring devices. Monitoring data may be collected continuously, or near continuously, or periodically, or in some combination (where some data is collected periodically and some data is collected continuously or near continuously).

Animal insurance parameters determination system 700 is configured to obtain at least a subset of the records, the subset being associated with a group of members of the animal population (block 810). In some cases, all of the records of the animal population are obtained by animal insurance parameters determination system 700. In other cases, the group of members can consist of a sub-population of the animal population, selected in accordance with one or more criteria, such as a location of the sub-population. For example: the sub-population can be the animals that are located within a given farm. It is to be noted that the animal population can be located in one or more geographical sites and/or within specific locations within these sites (e.g., enclosures, pasture, treatment areas, etc.).

The animals of the animal population can be for example: ruminating animals, livestock, swine, companion animals or any other type of non-human animal.

After obtaining the at least subset of the records, animal insurance parameters determination system 700 is further configured to determine one or more greenhouse gas emission affecting parameters based on the subset (block 820). The greenhouse gas emission affecting parameters can include one or more of the following parameters:
(a) Heat Detection Rate (HDR) calculated for the group of members, noting that HDR can be measured by dividing a length of a predetermined heat cycle (e.g. 21 days for dairy cows) with an actual average length of estrus of the animals of the animal population (noting that heat can be detected based on the monitoring data analysis, as discussed, for example by: (a) A. M. L. Madureira, B. F. Silper, T. A. Burnett, L. Polsky, L. H. Cruppe, D. M. Veira, J. L. M. Vasconcelos, R. L. A. Cerri, “Factors affecting expression of estrus measured by activity monitors and conception risk of lactating dairy cows”, Journal of Dairy Science, Volume 98, Issue 10, 2015, Pages 7003-7014, (b) R. C. Neves, K. E. Leslie, J. S. Walton, S. J. LeBlanc, “Reproductive performance with an automated activity monitoring system versus a synchronized breeding program”, Journal of Dairy Science, Volume 95, Issue 10, 2012, Pages 5683-5693, (c) S. Reith, S. Hoy, “Relationship between daily rumination time and estrus of dairy cows”, Journal of Dairy Science, Volume 95, Issue 11, 2012, Pages 6416-6420, (d) S. Reith, H. Brandt, S. Hoy, “Simultaneous analysis of activity and rumination time, based on collar-mounted sensor technology, of dairy cows over the peri-estrus period”, Livestock Science, Volume 170, 2014, Pages 219-227, and (e) S. P. M. Aungier, J. F. Roche, M. Sheehy, M. A. Crowe, “Effects of management and health on the use of activity monitoring for estrus detection in dairy cows”, Journal of Dairy Science, Volume 95, Issue 5, 2012, Pages 2452-2466, all of which are incorporated herein by reference).
(b) a conception rate calculated for the group of members, indicative of a ratio of conceptions to estruses of animals of the group.
(c) a health score calculated for the group of members. Before turning to the next parameter, it is to be noted that the health score can be determined as follows:

For the health score 120 calculation, the parameters that can be used can include, for example, the energy level of the given member and/or the time the given member ruminates. Rumination, energy levels, heavy breathing, chewing, core temperature, Image analysis to identify indicators of health problems and optionally additional and/or alternative indicators from the member can determine if the member has health disorders (e.g., illness and/or injury, etc.). The health score 120 calculation can be based on the absolute values of these parameters, on changes of these monitored parameters over time, relative values of these parameters (such as percentages, etc.) or on a combination thereof.

The health score 120 for the given member can be determined, for example, based on the consistency of the values of at least some of the parameters over time (e.g., their consistency over a rolling average for a given time period, such as a ten-day rolling average). In some cases, the consistency of the values can be measured against reference parameters, for example: reference parameters that are measured from reference animals. Actual members of the animal population (other than the given member) or from another population can be used as reference animals. Historical data can also be used as reference. In some cases, the reference can be a theoretical reference of how a hypothetical reference animal should behave.

Additionally, or alternatively, the health score 120 for a given member of the animal population is determined based on criteria of basic health and functioning of the given member, including, specifically, freedom from disease and injury. In some embodiments, health score 120 can be calculated based, at least in part, on identifying symptoms that can be associated with health-related issues (e.g., illness, injury, etc.). In some cases, the health score 120 is determined based on finding statistically significant correlations between the identified symptoms and illnesses or injuries. In some cases, the strengths of such correlations between the symptoms that are indicative of illness and/or injuries is also used in order to determine the health score 120.

Those symptoms can be identified by monitoring rumination and/or energy levels of an individual member, of a group of members of the animal population or, of the entire animal population. Additionally, or alternatively, the symptoms can be identified by based, at least in part, on monitoring visual indicators associated with health problems and/or monitoring breathing patterns of an individual member, of a group of members of the animal population or, of the entire animal population.

Energy level can be measured, for example, by detecting movements of a given member over a given time period (e.g., a day) and deducing the amount of energy required from the given member to perform these movements (optionally based on specific characteristics of the given animal). The movements can be detected by monitoring an acceleration signal obtained for example from an accelerometer comprised within a monitoring device attached to the given member.

Rumination can be measured, for example, by detecting ruminating activity of a given member over a given time period (e.g., a day). This can be achieved by analyzing acceleration signal obtained for example from an accelerometer comprised within a monitoring device attached to the given member and detecting signals that correlate to reference signals that are known to be associated with rumination activity.

Breathing patterns can be determined, for example, by detecting various breathing patterns of a given member over a given time period (e.g., a day). This can be achieved by analyzing acceleration signal obtained for example from an accelerometer comprised within a monitoring device attached to the given member and detecting signals that correlate to reference signals that are known to be associated with specific breathing patterns. This can enable determining whether the given animal demonstrates suspicious behavior (e.g., if it is breathing heavily more than usual and/or over a predetermined threshold, etc.).

Visual indicators associated with health problems can be identified using image analysis of images and/or videos of the members of the animal population. For example, scratching or irritation that are correlated with morbidity symptoms can be identified using image and/or video analysis.

It is to be noted that these are mere examples for determination of symptoms that can be associated with health-related issues, and additional and/or alternative data can be used to determine the above indicated symptoms, or other symptoms that can be associated with health-related issues.

The health score 120 of the members of the animal population can be used for calculating a health index indicative of the overall health level of the members of the animal population. In some cases, the health index can be used for identifying morbidity levels in the animal population. For example, a health index showing that more than a threshold (e.g., around 5%) of the animals in the animal population are having health scores 120 below a threshold can be an indication of a developing health problem within the animal population. A health index showing that more than a second threshold (e.g., around 10%) of the animal population are having health scores 120 below a threshold can be an indication of a prevailing health problem. A health index showing that more than a third threshold (e.g., around 20%) of the animal population are having health scores 120 below a threshold can be indicative of a pandemic.

It is to be noted that analysis of the health scores 120 can enable identification of given animals of the animal population that have a certain health issue (e.g., illness, lameness, estrus, etc.). In some cases, analysis of the health index (generated based on the health scores 120 of the members of the animal population) can enable, at the population level, identifying whether the overall health level of the animal population is above an acceptable predetermined health threshold.

It is to be further noted, as further detailed herein, inter alia with reference to FIGS. 10 and 11, that in some cases, the health scores 120 and/or components thereof (e.g. rumination time) can be used in the estimation of greenhouse gas emission.

Returning to the next parameter:
(d) rumination consistency, (also referred to herein as consistency of Rumination Variability (RV)), calculated for the group of members, based on the monitoring data; Rumination consistency is indicative of a level of consistency (e.g., on a scale of 1-100) of rumination time of the members of the group over a period of time; In a certain example (non-limiting), the RV can be calculated by determining daily rumination averages of all members of the group (noting that rumination time of each member can be determined using the monitoring data, as known in the art) for a sliding window of 10 days, and then calculating a variability between the calculated averages calculated in the 10 days sliding window;
(e) rumination time heterogeneity calculated for the group of members, based on the monitoring data; Rumination time heterogeneity is indicative of the heterogeneity of rumination time of the members of the group; In a certain example, the rumination time heterogeneity can be calculated by determining a standard deviation between daily rumination times of the members of the animal population (noting that rumination time of each member can be determined using the monitoring data, as known in the art); or
(f) average grazing time calculated for the group of members during a given time period, based on the monitoring data; It is to be noted that grazing farms in which animals are fed mainly by grazing in pasture, are less effective in terms of milk production when compared to farms in which the animals are mainly fed with food compounds that are designed to increase the animals milk yield, and thus the GHG emission in such farms is not optimal as shifting to such compounds will result in less GHG emission per milk output for the same animal population. It is to be noted that a certain amount of grazing may not have a negative effect on the animal's milk yield (in some cases, even up to 50% of the feeding time spent grazing may not have a negative effect on milk yield).

When looking at HDR, some farms demonstrate an HDR of less than 60%. When compared with such farms, farms that demonstrate an HDR of 60%-70% demonstrate an improvement of about 10% in methane output (about 10% decrease in methane output per milk unit). Farms that demonstrate an HDR higher than 70% demonstrate an additional improvement of about 10% in methane output (and 20% improvement when compared with farms that demonstrate an HDR of less than 60%). Similarly, when looking at the conception rate, the higher the conception rate is, the lower the methane output is per milk unit.

In the context of the HDR and conception rate, attention is drawn to FIG. 10. As can be seen when looking at FIG. 10, there is a connection between HDR and conception rates within an animal population on the one hand and methane and ammonia output produced by that animal population on the other hand, so that the higher the estrus detection and conception rates are—the lower the methane output per milk unit is. In addition, it can be appreciated from this figure that higher milk yielding cows generate less GHG per milk unit when compared to lower milk yielding cows. The graph shown in FIG. 10, which illustrates the connection between HDR and conception rates of an animal population on the one hand and methane and ammonia output produced by the animal population on the other hand, is taken from P. C Garnsworthy, “The environmental impact of fertility in dairy cows: a modelling approach to predict methane and ammonia emissions”, Animal Feed Science and Technology, Volume 112, Issues 1-4, 2004, Pages 211-223, which is incorporated herein by reference.

When looking at the health score, it has been shown that Sub Clinical Ketosis (SCK) causes a direct milk loss of at least 5% of the milk production of dairy cows suffering from SCK. Some studies even demonstrate a direct milk loss of 7% of the milk production of dairy cows suffering from SCK (see P. F. Mostert, C. E. van Middelaar, E. A. M. Bokkers, I. J. M. de Boer, “The impact of subclinical ketosis in dairy cows on greenhouse gas emissions of milk production”, Journal of Cleaner Production, Volume 171, 2018, Pages 773-782). Current systems are able to identify about 90% of the SCK cases. Thus, even if we assume that a direct milk loss of the milk production of dairy cows suffering from SCK is only 5%, and only 4% will be saved (and not all 5%), the savings in terms of GHG emissions are considerable (due to the fact that milk yield loss is avoided, and thus the GHG emission per unit of milk produced is reduced).

When looking at rumination consistency, it has been shown that rumination is highly correlated with fiber feed component particle length and quality (see, for example, (a) R. E. Coon, T. F. Duffield, T. J. DeVries, “Effect of straw particle size on the behavior, health, and production of early-lactation dairy cows”, Journal of Dairy Science, Volume 101, Issue 7, 2018, Pages 6375-6387, (b) Coon R E, Duffield T F, DeVries T J., “Short communication: Risk of subacute ruminal acidosis affects the feed sorting behavior and milk production of early lactation cows”, Journal of Dairy Science, Volume 102, Issue 1, 2019, pages 652-659, and (c) G. Adin, R. Solomon, M. Nikbachat, A. Zenou, E. Yosef, A. Brosh, A. Shabtay, S. J. Mabjeesh, I. Halachmi, J. Miron, “Effect of feeding cows in early lactation with diets differing in roughage-neutral detergent fiber content on intake behavior, rumination, and milk production”, Journal of Dairy Science, Volume 92, Issue 7, 2009, Pages 3364-3373). It has also been shown that inconsistency in fiber availability in the animal's feed can contribute to 3% reduction in milk production in a population of dairy cows (see A. D. Sova, S. J. LeBlanc, B. W. McBride, T. J. DeVries, “Accuracy and precision of total mixed rations fed on commercial dairy farms”, Journal of Dairy Science, Volume 97, Issue 1, 2014, Pages 562-571).

Some farms demonstrate relatively poor rumination consistency in which rumination changes by over 20 minutes per day on average. Other farms demonstrate a rumination consistency between 10-20 minutes per day on average. Other farms demonstrate a rumination consistency of less than 10 minutes per day on average. In view of the fact that lower rumination consistency is attributable to feeding deficiency, and thus to lower milk yield (as detailed herein above), it is expected, in terms of CO₂ output, that (a) farms that demonstrate a rumination consistency between 10-20 minutes per day on average will demonstrate a decrease of 2% in CO₂ output per milk unit when compared with farms that demonstrate rumination consistency in which rumination changes by over 20 minutes per day on average, and (b) farms that demonstrate a rumination consistency of less than 10 minutes per day on average will demonstrate a decrease of 3% in CO₂ output per milk unit when compared with farms that demonstrate rumination consistency in which rumination changes by over 20 minutes per day on average.

When looking at rumination heterogeneity, assuming an expected rumination variability is 16%, as is the case when dealing with dairy cows (as shown in K. A. Beauchemin, “Invited review: Current perspectives on eating and rumination activity in dairy cows”, Journal of Dairy Science, Volume 101, Issue 6, 2018, Pages 4762-4784), which translates to 80 minutes per day (based on 500 rumination minutes per day, according to the calculation of 500*0.16=80 minutes per day), some farms demonstrate relatively poor rumination heterogeneity in which the standard deviation of rumination between the animals is higher than 100 minutes per day. Other farms demonstrate a rumination heterogeneity in which the standard deviation of rumination between the animals is between 80-100 minutes per day. Other farms demonstrate a rumination heterogeneity in which the standard deviation of rumination between the animals is less than 80 minutes per day.

In terms of CO₂ output, farms that demonstrate a rumination heterogeneity in which the standard deviation of rumination between the animals is between 80-100 minutes per day demonstrate a decrease of 2% when compared with farms that demonstrate a standard deviation of rumination between the animals is higher than 100 minutes per day. Farms that demonstrate a rumination heterogeneity in which the standard deviation of rumination between the animals is less than 80 minutes per day demonstrate a decrease of 3% when compared with farms that demonstrate a standard deviation of rumination between the animals is higher than 100 minutes per day.

In this context, attention is drawn to FIG. 11, a schematic illustration of a relationship between yield of methane and carbon dioxide per units of milk, taken from an Watt et al.'s article “Differential rumination, intake, and enteric methane production of dairy cows in a pasture-based automatic milking system”, published on Journal of Dairy Science, Volume 98, Issue 10, October 2015, Pages 7248-7263. In the graph, the relationship between yield of methane (QCH4/milk; circles) and carbon dioxide (QCO2/milk; squares) per units of milk and the milk production of a population of dairy cows on a pasture-based automatic milking system is shown. Values for dairy cows classified as High Ruminating (HR, shown in solid symbols within the graph) or Low Ruminating (LR, shown in open symbols within the graph) are shown with corresponding exponential functions showing reductions of QCH4/milk (in the solid line) and of QCO2/milk (in the dashed line) as a function of milk production, across the animal population. Looking at the graph, it can be appreciated that there is a connection between the rumination heterogeneity and the methane and carbon dioxide emissions per milk unit, as low ruminating animals produce, on average, less milk (and thus more methane and carbon dioxide per milk unit), than high ruminating animals. The graph further indicates that the higher the milk yield of the animals is, the lower the methane and carbon dioxide emissions per milk unit are.

It is to be noted that these are exemplary parameters only, and in some cases, additional and/or alternative greenhouse gas emission affecting parameters can be calculated/obtained and used. In some cases, such additional and/or alternative greenhouse gas emission affecting parameters may include environmental parameters such as an ambient temperature in the environment of the animal population (as excessive heat in the animal population's environment can have a negative effect on the animal's milk yield (thus resulting in higher GHG emission per milk unit), etc. It is to be noted that every parameter that has an effect on milk yield of a given animal population can be taken into account, as an increase of milk yield of the animal population is likely to lead to an improved GHG emission per milk unit ratio. Animal insurance parameters determination system 700 is configured to utilize the greenhouse gas emission affecting parameters in order to estimate an amount of greenhouse gas emission in the environment housing the animal population (block 830).

In some cases, the estimation can be based on comparison of the greenhouse gas emission affecting parameters to a given baseline. The given baseline can optionally be determined using direct measurement of greenhouse gas emission via greenhouse gas emission measuring equipment, acquired from the environment housing the animal population, or from another environment having known properties (e.g. size, size of animal population, or any other property that can be used to adapt the baseline to the environment housing the animal population). In some cases, the baseline can be geographical location specific (e.g., as geographical characteristics such as weather can have an effect on greenhouse gas emissions). In some cases, the baseline is determined based on all, or some, of the historical values of the greenhouse gas emission affecting parameters determined at block 820. It is to be noted that in such cases, where the baseline is determined based on historical values of greenhouse gas emission affecting parameters, the baseline can be validated using direct measurement of greenhouse gas emission acquired using greenhouse gas emission measuring equipment, acquired from the environment housing the animal population. Such validation can be a one-time validation occurring at a certain point in time, or a periodical validation occurring once in a while (e.g., once a month, once a year, once every few years, or any other time interval, which is not necessarily constant between subsequent validations).

Animal insurance parameters determination system 700 is further configured to perform an action utilizing the estimated amount of greenhouse gas emission in the environment (block 840). In some cases, the action can be one or more of: (a) determining one or more insurance parameters for insuring at least one animal of the animal population, or the entire animal population, based on the estimated amount, (b) providing a user of the system with an indication of the estimated amount of greenhouse gas emission, or (c) suggesting one or more greenhouse gas emission reduction actions to be taken on the animal population, or part/s thereof, in order to reduce the greenhouse gas emission in the environment.

In some cases, the one or more insurance parameters can include one or more of: insurance price (which can be, for example, correlated to the GHG emissions so that when emissions increase the insurance price increases and vice versa), insurance period (which can be, for example, negatively correlated to the GHG emissions so that when emissions increase the insurance period decreases and vice versa), obligations to meet in order to be eligible for the insurance (e.g. a requirement to reduce the GHG emissions over time, optionally with milestones, etc.), etc.

The greenhouse gas emission reduction actions can include one or more of: (i) administering a treatment to the animal population, or part/s thereof, (ii) changing a temperature of an environment of the animal population, or part/s thereof, (iii) changing a feed of the animal population, or part/s thereof (e.g. in order to improve the rumination consistency), (iv) changing a schedule of the animal population, or part/s thereof, (v) improving the reproduction of the animal population (e.g. improving the HDR, the conception rate, etc.), (vi) improving the welfare of the animal population, (vii) improving husbandry conditions, etc.

In some case, although not shown in the figure, following acting upon the recommendations, or performing any other action that the user deems fit, the user can update the animal insurance parameters determination system 700 with an indication of the actions taken. In such cases, the animal insurance parameters determination system 700 can be configured to repeat the animal insurance parameters determination process 800 following collection of additional data from the monitoring devices in order to estimate the effect of the actions made by the user on the GHG emission of the animal population. In this manner, the system can be used to evaluate the impact of various actions taken on GHG emissions. In some cases, changes to the GHG emission of the animal population can also affect the insurance parameters, some of which can optionally dynamically change as a result of such changes (e.g. reduction in GHG emission may lead to reduction in the insurance price, and vice versa, etc.).

It is to be noted that having the ability to provide a user of the system (e.g. a farmer) with an indication of the estimated amount of greenhouse gas emission generated by the animal population (e.g. the animals in the farmers farm) in real-time or near real-time is extremely advantageous, as it can enable the user to take action when there is a need. Automatic estimation of the greenhouse gas emission, using the teachings herein, can be made without expensive and hard to operate equipment, and optionally in real time or near real-time, as opposed to existing solutions which require human auditing or direct measurements acquired by dedicated instrumentation.

As indicated herein, human auditors base their GHG emission estimation on data collected in a slow and manual process that is prone to errors. As part of such process, the human auditors visit and visually inspect the facilities housing the animals, and gather various types of data on the animal population itself. Based at least in part on the human auditor's subjective impression, the human auditor estimates the GHG emissions in the audited environment. One exemplary problem of subjectivity of an auditor is its first impression of an animal facility. For a human auditor, it may be hard to shake off a first impression, or a previous impression (not necessarily first), of a facility. Such first impression can lead to erroneous assessment of GHG emissions in such facility. For example, if the animal facility makes a good first impression, the auditor may miss out on other problems, or give them lesser weight, or vice versa. It can be appreciated that efforts are made to train auditors to maintain standard levels of evaluation, however interobserver reliability can be low despite training of the human auditors. Even in those cases where auditors undergo identical training, that training needs to be continuous to hold high standards of consistency, and even in these cases, the subjectiveness bias is not, and cannot, be completely irradicated.

Thus, current GHG emission estimation methods are prone to the subjectivity of the auditor. On top of that, they are also sensitive to the data collection method, sensitive to the timing circumstances, and frequency of the inspection (lighting conditions, weather conditions, etc.), and require long gathering and processing times.

In addition, GHG emission estimations may vary between auditors, and between audits, because they are not produced in real-time, nor are they based on continuous monitoring. Human auditing is not based on continuous, or near continuous, monitoring of various parameters (e.g., parameters relating to the animals' environment, the animals' health measures, animal treatments, the weather, etc.), inter alia because it is impossible for a human to track such amounts of data, let alone track such amounts of data in a manner that will enable evaluating GHG emissions in the required speed. To the contrary, having an automatic mechanism for estimating GHG emissions, optionally in real-time or near real-time, continuously or near continuously, can enable a much completer and more accurate GHG emission estimation that is not dependent on outdated or no-longer relevant data.

As mentioned above, GHG emissions can be measured directly using dedicated instrumentation, however such instrumentation is usually unavailable for livestock growers to continuously and directly measure greenhouse gas emissions in the environment in which their livestock is grown. Direct measurement of GHG emissions is also an extremely difficult task to perform when the animals are not within a housing in which the dedicated instrumentation can yield accurate results. For example, dedicated instrumentation cannot provide an accurate measurement when the animal population is grazing in pasture because the actual gas emission generated by the animal population cannot be captured in an open area such as this (e.g. as the natural emissions generated by animals are below the sensitivity level of standard GHG measurement equipment).

Further, the system and method disclosed herein can provide the animal caregiver and/or a regulator (such as a governmental organization) with information that can be used to take action in real or near real time to improve greenhouse gas emissions, such as by obtaining treatments for veterinary or health issues such as: infectious diseases, ectoparasites, and/or reproductive issues. This can also have an effect on insurance plans that can be offered to the animal caregiver, and/or on changes to existing insurance plans.

Moreover, based on the system and method disclosed herein, benchmarks can be determined for one or more locations or facilities of animal populations. These benchmarks can be used as references over time to automatically determine the adherence of the facilities or locations with an expected level of greenhouse gas emissions. These benchmarks can be measured and validated, or adjusted if needed, optionally continuously. The benchmarks can also be used as part of the determination of the insurance parameters.

The greenhouse gas emission estimation disclosed herein can be used to set standards (optionally industry wide standards), benchmark between different animal populations and/or between groups of members of one or more animal populations, and provide management or veterinarian support, optionally in real time, to reduce greenhouse gas emissions. The greenhouse gas emission estimation disclosed herein can also be used by insurance and/or finance providers to evaluate risks in insuring and/or financing related to the animal population, and to determine one or more insurance parameters, as disclosed herein.

For example, the greenhouse gas emission estimation disclosed herein can be used to set standards for greenhouse gas emissions for various types of animal caregivers, whether institutional (such as farms in which cows, swine, equine or any other type of animal is maintained, for slaughter, milk production or for any other purpose) or private animal owners (such as pet owners). Having such standards can enable regulation (enabling governments or other regulators make sure that greenhouse gas emission meets the required standards, and quickly identify any irregularity), value and/or risk estimation (enabling insurers, lenders or other type of financial institutions, trade associations, customers, or any other entity that is interested in valuation and/or risk estimation of the animals or their products, to more accurately evaluate the animals or their products such as milk, meat, etc.), and more. For example, the improved objectivity, consistency, frequency, and reliability of the greenhouse gas emission estimation and reduction systems and methods descried herein allows for more accurate and precise comparison between disparate farms/animal facilities, which is one of the advantages that enables entities to generate effective standard setting, risk estimation regulation, etc.

In some cases, the greenhouse gas emission estimation can also be used as a measure based on which a certification of greenhouse gas emissions can be provided, similarly to the Marine Stewardship Council (MSC) Certification used in the fishing industry.

It is to be noted that the ability of the system disclosed herein to monitor the greenhouse gas emission estimation associated with animal populations in a continuous (or near continuous) reliable, consistent, and objective manner, is extremely powerful and important. The monitoring described herein enables all stakeholders (animal caregivers, regulators, financial institutions, trade associations, milk processors, meat processors, retail chains, end-customers, or any other entity that may have an interest in the animals' welfare) to receive relevant information for making decisions in real-time, or almost in real-time (e.g., within milliseconds, seconds, minutes, hours or days) based on continuously, or near continuously, updated reliable, consistent, and objective information (as opposed to existing solutions in which such information is unavailable and requires transferring expensive and hard to operate greenhouse gas emission measurement equipment).

A few non-limiting examples for uses of the greenhouse gas emission estimation according to the presently disclosed subject matter: financial institutions can evaluate risks based on relevant information indicative of the greenhouse gas emission estimation at the time they make decisions, and can determine insurance parameters based thereon; Regulators can act upon a detected irregularity when it occurs and not in retrospect; retail chains, milk processors, meat processors, end-customers, or any other consumers of products of the animal population can get information of the greenhouse gas emission estimation of the animal population from which the products originate; diary and/or meat producers can compare farms based on the greenhouse gas emission estimation, so they can make informed purchasing decisions.

It is important to note that the greenhouse gas emission estimation and reduction according to the teachings herein is determined based, at least in part, on direct analysis of the animals behavioural and physiological signals and not based on human interpretation or measurement by dedicated greenhouse gas emission measurement machinery.

The greenhouse gas emission estimation can be also used by regulation entities or sourcing companies for monitoring regulated or supplier farms, develop procedures with the animal care giver they regulate or source from, manage public and customer opinion and ensure they maintain transparency around their brand name and brand value.

It is to be noted, with reference to FIG. 9, that some of the blocks can be integrated into a consolidated block or can be broken down to a few blocks and/or other blocks may be added. It should be also noted that whilst the flow diagram is described also with reference to the system elements that realizes them, this is by no means binding, and the blocks can be performed by elements other than those described herein.

It is to be understood that the presently disclosed subject matter is not limited in its application to the details set forth in the description contained herein or illustrated in the drawings. The presently disclosed subject matter is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the present presently disclosed subject matter.

It will also be understood that the system according to the presently disclosed subject matter can be implemented, at least partly, as a suitably programmed computer. Likewise, the presently disclosed subject matter contemplates a computer program being readable by a computer for executing the disclosed methods. The presently disclosed subject matter further contemplates a machine-readable memory tangibly embodying a program of instructions executable by the machine for executing the disclosed methods.

1. A system for determining insurance parameters, the system comprising: one or more monitoring devices configured to monitor parameters of members of an animal population;
a data repository comprising two or more records, each of the records (i) being associated with a respective member of the members, and (ii) including one or more monitored parameters of the respective member as monitored by at least one of the monitoring devices over time; and
a processing circuitry configured to:
obtain at least a subset of the records, the subset being associated with a group of given members of the members;
determine one or more greenhouse gas emission affecting parameters based on the subset, wherein at least one greenhouse gas emission affecting parameter of the greenhouse gas emission affecting parameters is determined based on one or more of the monitored parameters that are monitored for at least two members of the group of given members;
estimate an amount of greenhouse gas emission in an environment housing the animal population utilizing the greenhouse gas emission affecting parameters; and
determine one or more insurance parameters, for insuring at least one animal of the animal population, based on the estimated amount.

2. The system according to claim 1, wherein the insurance parameters include one or more of: insurance price, insurance period, or obligations to meet in order to be eligible for insurance.

3. The system according to claim 1, wherein the estimation is based on comparison of the greenhouse gas emission affecting parameters to a given baseline.

4. The system according to claim 3, wherein the baseline is geographical location specific.

5. The system according to claim 3, wherein the baseline is determined based on historical values of the greenhouse gas emission affecting parameters.

6. The system according to claim 3, wherein the baseline is determined using greenhouse gas emission measurements acquired using greenhouse gas emission measuring equipment.

7. The system according to claim 6, wherein the greenhouse gas emission measurements are acquired from the environment.

8.-10. (canceled)

11. The system of claim 10, wherein the processing circuitry is further configured to calculate, based on the subset of the records, for each given member of the group of given members, at least one of: (a) an animal health score indicative of a health state of the respective member, (b) an animal natural living score, indicative of compliance of a behavior pattern of the respective member with a desired natural behavior pattern, or (c) an animal affectivity/happiness score, indicative of compliance of an affectivity/happiness measure of the respective member with a desired affectivity/happiness measure.

12.-21. (canceled)

22. The system according to claim 1, wherein at least some of the records further include descriptive data associated with the respective member, wherein the descriptive data is not obtained from the monitoring devices.

23. The system according to claim 22, wherein the descriptive data includes one or more of: age of the respective member, sex of the respective member, treatment history of the respective member, or genetic information associated with the respective member.

24.-25. (canceled)

26. A method for determining insurance parameters, the method comprising: obtaining, by a processing circuitry, at least a subset of two or more records, each of the records (i) being associated with a respective member of members of an animal population, and (ii) including one or more monitored parameters of the respective member as monitored over time by at least one of a group of one or more monitoring devices configured to monitor parameters of the members of the animal population, wherein the subset is associated with a first group of given members of the members;
determining, by the processing circuitry, one or more greenhouse gas emission affecting parameters based on the subset, wherein at least one greenhouse gas emission affecting parameter of the greenhouse gas emission affecting parameters is determined based on one or more of the monitored parameters that are monitored for at least two members of the first group of given members;
estimating, by the processing circuitry, an amount of greenhouse gas emission in an environment housing the animal population utilizing the greenhouse gas emission affecting parameters; and
determining, by the processing circuitry, one or more insurance parameters, for insuring at least one animal of the animal population, based on the estimated amount.

27. The method according to claim 26, wherein the insurance parameters include one or more of: insurance price, insurance period, or obligations to meet in order to be eligible for insurance.

28. The method according to claim 26, wherein the estimating is based on comparison of the greenhouse gas emission affecting parameters to a given baseline.

29. The method according to claim 28, wherein the baseline is geographical location specific.

30. The method according to claim 28, wherein the baseline is determined based on historical values of the greenhouse gas emission affecting parameters.

31. The method according to claim 28, wherein the baseline is determined using greenhouse gas emission measurements acquired using greenhouse gas emission measuring equipment.

32.-49. (canceled)

50. A non-transitory computer readable storage medium having computer readable program code embodied therewith, the computer readable program code, executable by at least one processing circuitry of a computer to perform a method for determining insurance parameters, the method comprising:
obtaining, by the processing circuitry, at least a subset of two or more records, each of the records (i) being associated with a respective member of members of an animal population, and (ii) including one or more monitored parameters of the respective member as monitored over time by at least one of a group of one or more monitoring devices configured to monitor parameters of the members of the animal population, wherein the subset is associated with a first group of given members of the members;
determining, by the processing circuitry, one or more greenhouse gas emission affecting parameters based on the subset, wherein at least one greenhouse gas emission affecting parameter of the greenhouse gas emission affecting parameters is determined based on one or more of the monitored parameters that are monitored for at least two members of the first group of given members;
estimating, by the processing circuitry, an amount of greenhouse gas emission in an environment housing the animal population utilizing the greenhouse gas emission affecting parameters; and
determining, by the processing circuitry, one or more insurance parameters, for insuring at least one animal of the animal population, based on the estimated amount.

51. The system of claim 8 wherein the processing circuity is configured to determine one or more insurance parameters, for insuring at least one animal of the animal population, based on the estimated amount of greenhouse gas emission, the animal health score, the animal natural living score, and the animal affectivity/happiness score.

52. The system of claim 8 wherein the affectivity/happiness measure is determined based on one or more of the following monitored parameters, being affectivity/happiness parameters: (1) respiration level of the given member, (2) percentage of rumination time within a time period of the given member, or (3) percentage of feeding time within a time period of the given member.

53. The system of claim 8 wherein the health score is calculated based on the animal health scores calculated for the group of given members; and the welfare score is calculated based on the at least one of: (a) the health score, (b) the animal natural living scores calculated for the group of given members, or (c) the animal affectivity/happiness scores calculated for the group of given members.
